SEQUENCE LISTING (13 sequences)

SEQ ID NO: 1 - DNA, 1071 bp, Homo sapiens
  source 1..1071: mol_type = other DNA
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 2 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 3 - DNA, 1074 bp, Mus musculus
  source 1..1074: mol_type = other DNA
atgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattag

SEQ ID NO: 4 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 5 - DNA, 1053 bp, Mus musculus
  source 1..1053: mol_type = other DNA
gcagtgtggagacacacccactttccccaagggctcctgctcccccaagtgatccccttatcctccgtgctaagatgacaccgaggttgcagtccttacctttgaaagcagccacaagggcgtgggggtgcacacctttaatcccagcactcgggaggcagaggcaggcagatttctgagttcgagaccagcctggtctacaaagtgaattccaggacagccagggctatacagagaaaccctgtcttgaaaaaaaaagagaaagaaaaaagaaaaaaaaaaatgaaagcagccacatctaaggactacgtggcacaggagagggtgagtccctgagagttcagctgctgccctgtctgttcctgtaaatggcagtggggtcatgggaaagtgaaggggctcaaggtattggacacttccaggataatcttttggacgcctcaccctgtgccaggaccaaggctgagcttggcaggctcagaacagggtgtcctgttcttccctgtctaaaacattcactctcagcttgctcacccttccccagacaaggaagctgcacagggtctggtgttcagatggctttggcttacagcaggtgtgggtgtggggtaggaggcagggggtaggggtgggggaagcctgtactatactcactatcctgtttctgaccctctaggactcctacagggttatgggagtggacaggcagtccagatctgagctgctgacccacaagcagtgccctgtgcctgccagaatccaaagccctgggaatgtccctctggtccccctctgtcccctgcagcccttcctgttgctcaaccttgcacagttccgacctgggggagagagggacagaaatcttgccaagtatttcaacagaatgtactggcaattacttcatggcttcctggacttggtaaaggatggactaccccgcccaacaggggggctggcagccaggtaggcccataaaaagcccgctggggagtcctcctcactctctgctcttcctcctccagcacacatcagacctagtagctgtggaaacca

SEQ ID NO: 6 - DNA, 1672 bp, Homo sapiens
  source 1..1672: mol_type = other DNA
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 7 - DNA, 1610 bp, Mus musculus
  source 1..1610: mol_type = other DNA
aactgagagtggaggggcacagaagagcccaagaggctccttaggttgtgtggagggtacaatatgtttgggctgagcaacccagagccagactttgtctggctggtaagagacagaggtgcctgctatcacaatccaagggtctgcttgaggcagagccagtgcaaaggatgtggttagagccagcctggtgtactgaagaggggcgaagagcttgagtaaggagtctcagcggtggtttgagaggcagggtggttaatggagtagctgcaggggagaatccttgggagggagcctgcaggacagagctttggtcaggaagtgatgggcatgtcactggaccctgtattgtctctgacttttctcaagtaggacaatgactctgcccagggagggggtctgtgacaaggtggaagggccagaggagaacttctgagaagaaaaccagaggccgtgaagaggtgggaagggcatgggattcagaacctcaggcccaccaggacacaaccccaggtccacagcagatgggtgaccttgcatgtctcagtcaccagcattgtgctccttgcttatcacgcttgggtgaaggaaatgacccaaatagcataaagcctgaaggccgggactaggccagctagggcttgcccttcccttcccagctgcactttccataggtcccaccttcagcagattagacccgcctcctgcttcctgcctccttgcctcctcactcatgggtctatgcccacctccagtctcgggactgaggctcactgaagtcccatcgaggtctggtctggtgaatcagcggctggctctgggccctgggcgaccagttaggttccgggcatgctaggcaatgaactctacccggaattgggggtgcggggaggcggggaggtctccaacccagccttttgaggacgtgcctgtcgctgcacggtgctttttatagacgatggtggcccattttgcagaagggaaagccggagccctctggggagcaaggtccccgcaaatggacggatgacctgagcttggttctgccagtccacttcccaaatccctcaccccattctagggactagggaaagatctcctgattggtcatatctgggggcctggccggagggcctcctatgattggagagatctaggctgggcgggccctagagcccgcctcttctctgcctggaggaggagcactgaccctaaccctctctgcacaagacccgagcttgtgcgcccttctgggagcttgctgcccctgtgctgactgctgacagctgactgacgctcgcagctagcaggtacttctgggttgctagcccagagccctgggccggtgaccctgttttccctacttcccgtctttgaccttgggtaagtttctttttcttttgtttttgagagaggcacccagatcctctccactacaggcagccgctgaaccttggatcctcagctcctgccctgggaactacagttcctgccctttttttcccaccttgagggaggttttccctgagtagcttcgactatcctggaacaagctttgtagaccagcctgggtctccggagagttgggattaaaggcgtgcaccaccacc

SEQ ID NO: 8 - DNA, 1387 bp, Homo sapiens
  source 1..1387: mol_type = other DNA
ctctggtttcaagaccaatactcataacccccacatggaccaggcaccatcacacctgagcactgcacttagggtcaaagacctggccccacatctcagcagctatgtagactagctccagtcccttaatctctctcagcctcagtttcttcatctgcaaaacaggtctcagtttcgttgcaaagtatgaagtgctgggctgttactggtcaaagggaagagctgggaagagggtgcaaggtggggttgggctggagatgggctggagcagatagatggagggacctgaatggaggaagtaaaccaaggcccggtaacattgggactggacagagaacacgcagatcctctaggcaccggaagctaagtaacattgccctttctcctcctgtttgggactaggctgatgttgctgcctggaagggagccagcagaagggccccagcctgaagctgttaggtagaagccaaatccagggccagatttccaggaggcagcctcgggaagttgaaacacccggattcaggggtcaggaggcctgggcttctggcaccaaacggccagggacctactttccacctggagtcttgtaagagccactttcagcttgagctgcactttcgtcctccatgaaatgggggaggggatgctcctcacccaccttgcaaggttattttgaggcaaatgtcatggcgggactgagaattcttctgccctgcgaggaaatccagacatctctcccttacagacagggagactgaggtgaggcccttccaggcagagaaggtcactgttgcagccatgggcagtgccccacaggacctcgggtggtgcctctggagtctggagaagttcctaggggacctccgaggcaaagcagcccaaaagccgcctgtgagggtggctggtgtctgtccttcctcctaaggctggagtgtgcctgtggaggggtctcctgaactcccgcaaaggcagaaaggagggaagtaggggctgggacagttcatgcctcctccctgagggggtctcccgggctcggctcttggggccagagttcagggtgtctgggcctctctatgactttgttctaagtctttagggtggggctggggtctggcccagctgcaagggccccctcacccctgccccagagaggaacagccccgcacgggccctttaagaaggttgagggtgggggcaggtgggggagtccaagcctgaaacccgagcgggcgcgcgggtctgcgcctgccccgcccccggagttaagtgcgcggacacccggagccggcccgcgcccaggagcagagccgcgctcgctccactcagctcccagctcccaggactccgctggctcctcgcaagtcctgccgcccagcccgccggg

SEQ ID NO: 9 - DNA, 9232 bp, synthetic construct
  misc_feature 1..9232: note = vector construct
  source 1..9232: mol_type = other DNA
gatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcaataaaagagcccacaacccctcactcggggcgccagtcctccgattgactgagtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcgctagcggatccggccgcctcggccaccggtcgccaccatcgccaccatgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattaggtttaaacgcggccgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgagcttgttaacatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttccgacttgtggtctcgctgccttgggagggtctcctctgagtgattgactacccgtcagcgggggtcttcacatgcagcatgtatcaaaattaatttggttttttttcttaagtatttacattaaatggccatagttgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 10 - DNA, 987 bp, Homo sapiens
  source 1..987: mol_type = other DNA
atgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaa

SEQ ID NO: 11 - protein, 328 aa, Homo sapiens
MTGVFDSLVADMHSTQIAASSTYHQHQQPPSGGGAGPGGNSSSSSSLHKPQESPTLPVSTATDSSYYTNQQHPAGGGGGGGSPYAHMGSYQYQASGLNNVPYSAKSSYDLGYTAAYTSYAPYGTSSSPANNEPEKEDLEPEIRIVNGKPKKVRKPRTIYSSFQLAALQRRFQKTQYLALPERAELAASLGLTQTQVKIWFQNRRSKFKKMWKSGEIPSEQHPGASASPPCASPPVSAPASWDFGVPQRMAGGGGPGSGGSGAGSSGSSPSSAASAFLGNYPWYHQTSGSASHLQATAPLLHPTQTPQPHHHHHHHGGGGAPVSAGTIF

SEQ ID NO: 12 - DNA, 999 bp, Mus musculus
  source 1..999: mol_type = other DNA
atgactggagtctttgacagtctggtggctgatatgcactcgacccagatcaccgcctccagcacgtaccaccagcaccagcagcccccgagcggtgcgggcgccggccctggcggcaacagcaacagcagcagcagcaacagcagcctgcacaagccccaggagtcgccaaccctcccggtgtccacggctacggacagcagctactacaccaaccagcagcacccggcgggcggcggcggcgggggggcctcgccctacgcgcacatgggctcctaccagtaccacgccagcggcctcaacaatgtctcctactccgccaaaagcagctacgacctgggctacaccgccgcgtacacctcctacgcgccctacggcaccagttcgtctccggtcaacaacgagccggacaaggaagaccttgagcctgaaatccgaatagtgaacgggaagccaaagaaagtccggaaaccacgcaccatctactccagtttccagctggcggcccttcaacgacgcttccagaagacccagtatctggccctgccagagcgagccgagctggcggcgtccctgggcctcacccaaactcaggtcaaaatctggttccagaaccgccgatccaagttcaagaagatgtggaaaagcggcgagatacccaccgagcagcaccctggagccagcgcttctcctccttgtgcctccccgccggtctcggcgccagcatcctgggacttcggcgcgccgcagcggatggctggcggcggcccgggcagcggaggcggcggtgcgggcagctctggctccagcccgagcagcgccgcctcggcctttctgggaaactacccgtggtaccaccaggcttcgggctccgcttcacacctgcaggccacagcgccacttctgcatccttcgcagactccgcaggcgcaccatcaccaccatcaccaccaccacgcaggcgggggcgccccggtgagcgcggggacgattttctaa

SEQ ID NO: 13 - protein, 332 aa, Mus musculus
MTGVFDSLVADMHSTQITASSTYHQHQQPPSGAGAGPGGNSNSSSSNSSLHKPQESPTLPVSTATDSSYYTNQQHPAGGGGGGASPYAHMGSYQYHASGLNNVSYSAKSSYDLGYTAAYTSYAPYGTSSSPVNNEPDKEDLEPEIRIVNGKPKKVRKPRTIYSSFQLAALQRRFQKTQYLALPERAELAASLGLTQTQVKIWFQNRRSKFKKMWKSGEIPTEQHPGASASPPCASPPVSAPASWDFGAPQRMAGGGPGSGGGGAGSSGSSPSSAASAFLGNYPWYHQASGSASHLQATAPLLHPSQTPQAHHHHHHHHHAGGGAPVSAGTIF

(19) United States
(12) Patent Application Publication
(10) Pub. No.: US 2024/0325488 A1
(43) Pub. Date: Oct. 3, 2024

(54) REGENERATING FUNCTIONAL NEURONS FOR TREATMENT OF HEMORRHAGIC STROKE

(71) Applicant: THE PENN STATE RESEARCH FOUNDATION, University Park, PA (US)

(72) Inventor: Gong CHEN, Guangzhou (CN)

(73) Assignee: THE PENN STATE RESEARCH FOUNDATION, University Park, PA (US)

(21) Appl. No.: 18/636,898

(22) Filed: Apr. 16, 2024

Related U.S. Application Data
(63) Continuation of application No. 17/073,037, filed on Oct. 16, 2020, now abandoned.
(60) Provisional application No. 62/916,706, filed on Oct. 17, 2019.

(51) Int. Cl. A61K 38/17; A61K 9/00; A61K 48/00; A61P 25/00

(52) U.S. Cl. CPC A61K 38/1703; A61K 9/0085; A61P 25/00; A61K 48/00

(57) ABSTRACT

This document provides methods and materials involved in treating mammals having had a hemorrhagic stroke. For example, methods and materials for administering a composition containing exogenous nucleic acid encoding a NeuroD1 polypeptide and exogenous nucleic acid encoding a Dlx2 polypeptide to a mammal having had a hemorrhagic stroke are provided.

### CROSS-REFERENCE TO RELATED APPLICATIONS

This application is a continuation of U.S. patent application Ser. No. 17/073,037, filed Oct. 16, 2020, which claims benefit of U.S. Provisional Application Ser. No. 62/916,706, filed on Oct. 17, 2019, the contents of these aforementioned applications being fully incorporated herein by reference.

### INCORPORATION OF SEQUENCE LISTING

A sequence listing contained in the file named P34798US02_SL_XML.xml, which is 36,864 bytes (measured in MS-Windows®) and created on Jun. 12, 2024, is filed electronically herewith and incorporated by reference in its entirety.

### BACKGROUND

### 1. Technical Field

This document relates to methods and materials involved in treating mammals having had a hemorrhagic stroke. For example, this document provides methods and materials for administering a composition containing exogenous nucleic acid encoding a NeuroD1 polypeptide (or a biologically active fragment thereof) and nucleic acid encoding a Dlx2 polypeptide (or a biologically active fragment thereof) to a mammal having had a hemorrhagic stroke.

### 2. Background Information

Stroke is a disease that affects the arteries leading to and within the brain. It is the number five cause of death and a leading cause of disability in the United States. A stroke occurs when a blood vessel that carries oxygen and nutrients to the brain is either blocked by a clot or bursts (Bonnard et al., Stroke, 50:1318-1324 (2019)). When that happens, part of the brain cannot get the blood (and oxygen) it needs, so it and brain cells die. Stroke can be caused either by a clot obstructing the flow of blood to the brain (called an ischemic stroke) or by a blood vessel rupturing and preventing blood flow to the brain (called a hemorrhagic stroke). A TIA (transient ischemic attack), or “mini stroke,” is caused by a temporary clot. Recent advances in neuroimaging, organized stroke care, dedicated Neuro-ICUs, and medical and surgical management have improved the management of hemorrhagic stroke. However, there remains a significant unmet need for treatment of patients having had a hemorrhagic stroke.

### SUMMARY

This document provides methods and materials involved in treating mammals having had a hemorrhagic stroke. For example, this document provides methods and materials for administering a composition containing exogenous nucleic acid encoding a NeuroD1 polypeptide (or a biologically active fragment thereof) and nucleic acid encoding a Dlx2 polypeptide (or a biologically active fragment thereof) to a mammal having had a hemorrhagic stroke.

In general, one aspect of this document features a method for (1) generating new glutamatergic neurons, (2) increasing survival of GABAergic neurons, (3) generating new non-reactive astrocytes, or (4) reducing the number of reactive astrocytes, in a mammal having had a hemorrhagic stroke and in need of (1), (2), (3), or (4). The method comprises (or consists essentially of or consists of) administering a composition comprising exogenous nucleic acid encoding a Neurogenic Differentiation 1 (NeuroD1) polypeptide or a biologically active fragment thereof and exogenous nucleic acid encoding a Distal-less homeobox 2 (Dlx2) polypeptide or a biologically active fragment thereof to the mammal. The mammal can be a human. The hemorrhagic stroke can be due to a condition selected from the group consisting of: ischemic stroke; physical injury; tumor; inflammation; infection; global ischemia as caused by cardiac arrest or severe hypotension (shock); hypoxic-ischemic encephalopathy as caused by hypoxia, hypoglycemia, or anemia; meningitis; and dehydration; or a combination of any two or more thereof. The administering step can comprise delivering an expression vector comprising a nucleic acid encoding a NeuroD1 polypeptide or a biologically active fragment thereof and an expression vector comprising a nucleic acid encoding a Dlx2 polypeptide or a biologically active fragment thereof to the location of the hemorrhagic stroke in the brain. The administering step can comprise delivering a recombinant viral expression vector comprising a nucleic acid encoding a NeuroD1 polypeptide or a biologically active fragment thereof and a recombinant viral expression vector comprising a nucleic acid encoding a Dlx2 polypeptide or a biologically active fragment thereof to the location of the hemorrhagic stroke in the brain.

The administering step can comprise delivering a recombinant adeno-associated virus expression vector comprising a nucleic acid encoding a NeuroD1 polypeptide or a biologically active fragment thereof and a recombinant adeno-associated virus expression vector comprising a nucleic acid encoding a Dlx2 polypeptide or a biologically active fragment thereof to the location of the hemorrhagic stroke in the brain. The administering step can comprise a stereotactic intracranial injection to the location of the hemorrhagic stroke in the brain. The administering step can further comprise administering the exogenous nucleic acid encoding a NeuroD1 polypeptide or a biologically active fragment thereof and exogenous nucleic acid encoding a Dlx2 polypeptide or a biologically active fragment thereof on one expression vector, one recombinant viral expression vector, or one recombinant adeno-associated virus expression vector. The composition can comprise about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof and a nucleic acid encoding a Dlx2 polypeptide or a biologically active fragment thereof. The composition can be injected in the brain of the mammal at a controlled flow rate of about 0.1 μL/minute to about 5 μL/minute.

In another aspect, this document features a method for (1) generating new GABAergic and glutamatergic neurons, (2) increasing survival of GABAergic and glutamatergic neurons, (3) generating new non-reactive astrocytes, or (4) reducing the number of reactive astrocytes, in a mammal having had a hemorrhagic stroke and in need of (1), (2), (3), or (4). The method comprises (or consists essentially of or consists of) administering a composition comprising exogenous nucleic acid encoding a Neurogenic Differentiation 1 (NeuroD1) polypeptide or a biologically active fragment thereof and exogenous nucleic acid encoding a Distal-less homeobox 2 (Dlx2) polypeptide or a biologically active fragment thereof to the mammal within 3 days of the hemorrhagic stroke. The mammal can be a human. The hemorrhagic stroke can be due to a condition selected from the group consisting of: bleeding in the brain; aneurysm; intracranial hematoma; subarachnoid hemorrhage; brain trauma; high blood pressure; weak blood vessels; malformation of blood vessels; ischemic stroke; physical injury; tumor; inflammation; infection; global ischemia as caused by cardiac arrest or severe hypotension (shock); hypoxic-ischemic encephalopathy as caused by hypoxia, hypoglycemia, or anemia; meningitis; and dehydration; or a combination of any two or more thereof. The administering step can comprise delivering an expression vector comprising a nucleic acid encoding a NeuroD1 polypeptide or a biologically active fragment thereof and an expression vector comprising a nucleic acid encoding a Dlx2 polypeptide or a biologically active fragment thereof to the location of the hemorrhagic stroke in the brain. The administering step can comprise delivering a recombinant viral expression vector comprising a nucleic acid encoding a NeuroD1 polypeptide or a biologically active fragment thereof and a recombinant viral expression vector comprising a nucleic acid encoding a Dlx2 polypeptide or a biologically active fragment thereof to the location of the hemorrhagic stroke in the brain. The administering step can comprise delivering a recombinant adeno-associated virus expression vector comprising a nucleic acid encoding a NeuroD1 polypeptide or a biologically active fragment thereof and a recombinant adeno-associated virus expression vector comprising a nucleic acid encoding a Dlx2 polypeptide or a biologically active fragment thereof to the location of the hemorrhagic stroke in the brain. The administering step can comprise a stereotactic intracranial injection to the location of the hemorrhagic stroke in the brain. The administering step can further comprise administering the exogenous nucleic acid encoding a NeuroD1 polypeptide or a biologically active fragment thereof and exogenous nucleic acid encoding a Dlx2 polypeptide or a biologically active fragment thereof on one expression vector, one recombinant viral expression vector, or one recombinant adeno-associated virus expression vector. The composition can comprise about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof and a nucleic acid encoding a Dlx2 polypeptide or a biologically active fragment thereof. The composition can be injected in the brain of the mammal at a controlled flow rate of about 0.1 μL/minute to about 5 μL/minute.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 comprises FIG. 1A and FIG. 1B. Iron evolution in collagenase-induced intracerebral hemorrhage (ICH) model. (FIG. 1A) At 1 and 2 days post stroke (dps), a very low level of ferric iron was detected via iron staining, and microglia started to migrate into the hematoma as determined by 3,3′-diaminobenzidine (DAB) staining. (FIG. 1B) At 8 and 29 dps, a high level of iron was detected in the injury core, intermingled with microglia, via iron staining, and astrocytes formed glia scar around injury core as determined by DAB staining. These results suggest that therapy no later than 2 days post stroke might be preferred.

FIG. 2 comprises FIG. 2A, FIG. 2B, FIG. 2C, FIG. 2D, FIG. 2E, FIG. 2F, FIG. 2G, FIG. 2H, FIG. 2I, FIG. 2J, FIG. 2K, FIG. 2L, FIG. 2M, FIG. 2N, FIG. 2O, and FIG. 2P. Conversion of astrocytes into neurons. FIG. 2A is a schematic showing the in vivo conversation of astrocytes being converted into functional neurons in a collagenase-induced ICH model. FIG. 2B is the experimental design used to confirm the in vivo conversion of reactive astrocytes to neurons in ICH model (intracerebral hemorrhage). ICH was induced with 0.2 μL of collagenase injected into the striatum. The control viruses were AAV5-GFAP-Cre (3×10¹¹; 1 μL)+AAV5-CAG-flex-GFP (3.4×10¹¹; 1 μL), and the treatment viruses were AAV5-GFAP-Cre (3×10¹¹; 1 μL)+AAV5-CAG-flex-ND1-GFP (4.55×10¹¹; 1 μL)+AAV5-CAG-flex-Dlx2-GFP (2.36×10¹²; 1 μL). FIG. 2C shows immunofluorescence staining for GFP, GFAP, and NeuN at 21 days post infection (dpi) with ND1 and Dlx2 viruses injected at 0 dps. Mild ICH was observed. GFAP signal was downregulated in the injury. Most of GFP⁺ cells showed neuronal morphologies. FIG. 2D shows immunofluorescence staining for GFP, GFAP, and NeuN at 21 days post infection with viruses designed to express ND1 and Dlx2 injected at 0 dps. Numbers 1, 2, and 3 refer to three nearby regions around the injury core. Most of GFP⁺ cells expressed NeuN. (FIG. 2E) At 19 days post induction with control or treatment viruses at 2 dps, many GFP⁺ cells showed neuronal morphologies in treatment side. FIG. 2F shows immunofluorescence staining for GFP, GFAP, and NeuN at 19 days post induction of viruses designed to express ND1 and Dlx2 at 2 dps. Numbers 1, 2, and 3 refer to three nearby regions around the injury core. Many GFP⁺ cells expressed NeuN. (FIG. 2G) At 17 days post induction with control or treatment viruses at 4 dps, fewer GFP⁺ cells showed neuronal morphologies in treatment side. FIG. 2H shows immunofluorescence staining for GFP, GFAP, and NeuN at 17 days post induction with viruses designed to express ND1 and Dlx2 at 4 dps. Numbers 1, 2, and 3 refer to three nearby regions around the injury core. Some GFP⁺ showed neuronal morphologies, while some are astrocytic. (FIG. 2I) At 14 days post induction with control or treatment viruses at 7 dps, GFP⁺ cells with neuronal morphologies are hardly observed. FIG. 2J shows immunofluorescence staining for GFP, GFAP, and NeuN at 14 days post induction with viruses designed to express ND1 and Dlx2 at 7 dps. Numbers 1, 2, and 3 refer to three nearby regions around the injury core. Almost all the GFP⁺ cells remained astrocytic morphologies. FIG. 2K shows immunofluorescence staining for GFP, GFAP, and NeuN for normal control, for virus control, and for treatment mice treated with viruses designed to express ND1 and Dlx2 at 0 dps, 2 dps, 4 dps, or 7 dps. Less GFP⁺ neurons, less neuronal density, and more reactive astrocytes were observed with the delay of injection time point. The optimal time point should not be longer than 2 dps. FIG. 2L shows the disappearance of GFAP observed in both treatment and control groups. FIG. 2M shows the disappearance of GFAP and NeuN signal at 21 days post induction with control viruses. FIG. 2N shows that while there was S100b signal in the GFAP-absent area in treatment mice, there was no S100b signal in the same area in control mice. (FIG. 2O) At 19 days post induction with control or treatment viruses at 2 dps, S100b signal appeared downregulated. FIG. 2P shows the downregulation of S100b in the treatment group, while S100b signal still showed the morphologies of reactive astrocytes in the control group.

FIG. 3 comprises FIG. 3A, FIG. 3B, FIG. 3C, FIG. 3D, FIG. 3E, FIG. 3F, FIG. 3G, and FIG. 3H. In vivo conversion of reactive astrocytes to neurons in ICH (long term). FIG. 3A is the experimental design used to confirm the in vivo conversion of reactive astrocytes to neurons in ICH (long term). ICH was induced with 0.35 μL of collagenase injected into the striatum. The control viruses were AAV5-GFAP-Cre (3×10¹¹; 1 μL)+AAV5-CAG-flex-GFP (3.4×10¹¹; 1 μL), and the treatment viruses were AAV5-GFAP-Cre (3×10¹¹; 1 μL)+AAV5-CAG-flex-ND1-GFP (4.55×10¹¹; 1 μL)+AAV5-CAG-flex-Dlx2-GFP (2.36×10¹²; 1 μL). FIG. 3B shows immunofluorescence staining for GFP, GFAP, and NeuN at 2 months post induction for mice treated with viruses designed to express ND1 and Dlx2 at 0 dps. Mild ICH was observed. Most of GFP⁺ cells are neuronal-like. FIG. 3C shows immunofluorescence staining for GFP, GFAP, and NeuN at 2 months post induction for mice treated with viruses designed to express ND1 and Dlx2 at 0 dps. Almost all the GFP⁺ cells expressed NeuN. FIG. 3D shows immunofluorescence staining for GFP, GFAP, and NeuN at 2 months post induction for mice treated with viruses designed to express ND1 and Dlx2 at 2 dps. Virus infection was not wide and was possibly too close to the ventricle. FIG. 3E shows immunofluorescence staining for GFP, GFAP, and NeuN at 2 months post induction for mice treated with viruses designed to express ND1 and Dlx2 at 7 dps. Mild ICH was observed. Many GFP⁺ neuronal-like cells were observed. FIG. 3F shows immunofluorescence staining for GFP, GFAP, and NeuN at 2 months post induction for mice treated with viruses designed to express ND1 and Dlx2 at 7 dps. A lower infection rate than that for 0 dps was observed. FIG. 3G shows immunofluorescence staining for GFP, GFAP, and NeuN at 2 months post induction for mice treated with control viruses at 0 dps. Many GFP⁺ cells were still astrocytes, while some GFP⁺ neurons were observed. FIG. 3H contains graphs plotting conversion (or leakage) rate (%) (left graph) and neuronal density (cell number×10⁴/mm³) (right graph) for mice treated as indicated. 2 dps-2M data was excluded due to inefficient virus infection. 0 dps-2M achieved the highest conversion rate (86%) and the highest neuronal density (147,000/mm³).

FIG. 4 comprises FIG. 4A, FIG. 4B, FIG. 4C, FIG. 4D, FIG. 4E, and FIG. 4F. AAV9-nonconcentrated 1.6 kb-GFAP-cre/flex system. FIG. 4A shows RFP staining at 19 days post induction with control viruses (AAV9-nonconcentrated-1.6 kb-GFAP-Cre+AAV9-flex-mCherry; left) or treatment viruses (AAV9-nonconcentrated-1.6 kb-GFAP-Cre+AAV9-flex-ND1-mCherry+AAV9-flex-Dlx2-mCherry; right) at 2 dps. In each case, 0.2 μL (0.03 Units) of collagenase was used to induce stroke. FIG. 4B shows immunofluorescence staining for NeuN, ND1, and RFP at 19 days post induction with viruses designed to express ND1 and Dlx2 at 2 dps. Not many neurons overexpressed ND1, but the signal of ND1 still was detected. FIG. 4C shows immunofluorescence staining for NeuN, Dlx2, and RFP at 19 days post induction with viruses designed to express ND1 and Dlx2 at 2 dps. Most neurons expressed Dlx2, and some of them did not exhibit RFP signal. FIG. 4D shows immunofluorescence staining for GFAP, RFP, and NeuN at 19 days post induction with control viruses or treatment viruses at 2 dps. RFP signal was decreased in the treatment group. High leakage still existed in AAV9-nonconcentrated cre. FIG. 4E shows immunofluorescence staining for Iba1 and RFP at 19 days post induction with control viruses or treatment viruses at 2 dps. Microglia in the treatment group seemed more reactive than those in the control group. FIG. 4F shows immunofluorescence staining for AQP4 (aquaporin 4) and RFP at 19 days post induction with control viruses or treatment viruses at 2 dps. A significant difference between control and treatment groups was not observed in AQP4 staining.

FIG. 5 comprises FIG. 5A, FIG. 5B, FIG. 5C, FIG. 5D, and FIG. 5E. AAV5-1.6 kb-GFAP-cre/flex system. FIG. 5A shows GFP staining at 19 days post induction with control viruses (AAV5-1.6 kb-GFAP-Cre+AAV5-flex-GFP; left) or treatment viruses (AAV5-1.6 kb-GFAP-Cre+AAV5-flex-ND1-GFP+AAV5-flex-Dlx2-GFP; right) at 2 dps. In each case, 0.2 μL (0.03 Units) of collagenase was used to induce stroke. FIG. 5B shows immunofluorescence staining for NeuN, GFP, ND1, and Dlx2 at 19 days post induction with viruses designed to express ND1 and Dlx2 at 2 dps. ND1 signal was not detected. Many neurons overexpressed Dlx2. In general, the signal was weaker than that observed with AAV9. FIG. 5C shows immunofluorescence staining for GFAP, GFP, and NeuN at 19 days post induction with control viruses or treatment viruses at 2 dps. The astrocytes in the treatment group appeared more reactive all over in the striatum. The astrocytes in the control group only appeared more reactive around the injury core. FIG. 5D shows immunofluorescence staining for Iba1 and GFP at 19 days post induction with control viruses or treatment viruses at 2 dps. In the control group, the reactive microglia were densely distributed in the injury core, while the reactive microglia in the treatment group also were observed in the peri-injury area. FIG. 5E shows immunofluorescence staining for AQP4 and RFP at 19 days post induction with control viruses or treatment viruses at 2 dps. The signal of AQP4 in the treatment group was potentially slightly stronger than that observed in the control group.

FIG. 6 comprises FIG. 6A, FIG. 6B, FIG. 6C, FIG. 6D, and FIG. 6E. FIG. 6A shows GFP, GFAP, and NeuN staining at 14 days post induction with a control virus (AAV5-1.6 kb-GFAP-Cre-5-flex-GFP) at 2 dps, which was induced with 0.5 μL (0.075 Units) of collagenase. FIG. 6B shows GFP, GFAP, and NeuN staining of a mild stroke at 14 days post induction with a treatment virus (AAV5-1.6 kb-GFAP-Cre-5-flex-ND1-GFP-5-flex-Dlx2-GFP) at 2 dps, which was induced with 0.5 μL (0.075 Units) of collagenase. FIG. 6C shows GFP, GFAP, and NeuN staining of a severe stroke at 14 days post induction with a treatment virus (AAV5-1.6 kb-GFAP-Cre-5-flex-ND1-GFP-5-flex-Dlx2-GFP) at 2 dps, which was induced with 0.5 μL (0.075 Units) of collagenase. FIG. 6D shows GFP, GFAP, and NeuN staining for a mild stroke at 2 months post induction with treatment viruses (AAV5-0.6 kb-GFAP-Cre+AAV5-flex-ND1-GFP+AAV5-flex-Dlx2-GFP) at 2 dps, which was induced with 0.5 μL (0.075 Units) of collagenase. MRI images were performed at 1 dps. FIG. 6E shows GFP, GFAP, and NeuN staining for a severe stroke at 2 months post induction with treatment viruses (AAV5-0.6 kb-GFAP-Cre+AAV5-flex-ND1-GFP+AAV5-flex-Dlx2-GFP) at 2 dps, which was induced with 0.5 μL (0.075 Units) of collagenase. MRI images were performed at 1 dps.

FIG. 7. Hematoma does not dissolve until 7 dps. RFP staining at 4 days post induction with control viruses (AAV9-nonconcentrated GFAP-Cre+AAV9-flex-mCherry) 2 dps, which was induced with 0.2 μL (0.03 Units) of collagenase. Virus will enter hematoma if it is injected in situ before 7 dps. The existence of hematoma might hinder the virus to target astrocytes.

FIG. 8. Proliferation peak of reactive astrocytes after ICH is around 7 dps. Astrocytes become reactive at 4 dps and start to form glia scar before 8 dps. See, also, Sukumari-Ramesh, et al., J. Neurotrauma, 29(18):2798-28044 (2012)).

FIG. 9. Besides virus injection time point, varying injury condition might also affect astrocyte to neuron conversion rates. GFP staining at 19, 17, or 14 days post induction with treatment viruses (AAV5-0.6 kb-GFAP-Cre+AAV5-flex-ND1-GFP+AAV5-flex-Dlx2-GFP) 2, 4, or 7 dps, respectively, which was induced with 0.2 μL (0.03 Units) of collagenase.

FIG. 10 comprises FIG. 10A, FIG. 10B, FIG. 10C, and FIG. 10D. Comparisons of astrocyte to neuron conversion rate in comparable injury conditions. Mouse #1 received treatment viruses (AAV5-0.6 kb-GFAP-Cre+AAV5-flex-ND1-GFP+AAV5-flex-Dlx2-GFP) 2 dps, which was induced with 0.325 μL (0.05 Units) collagenase. Mouse #2 received control viruses (AAV5-0.6 kb-GFAP-mCherry-Cre+AAV5-flex-GFP) in the left brain region 7 dps and treatment viruses (AAV5-0.6 kb-GFAP-Cre+AAV5-flex-ND1-GFP+AAV5-flex-Dlx2-GFP) in the right brain region 7 dps, which were induced in each side with 0.2 μL (0.03 Units) collagenase. FIG. 10A shows MRI scans for Mouse #1 (top) at 1 dps and Mouse #2 (bottom) at 3 dps. FIG. 10B shows GFP, GFAP, and NeuN staining of Mouse #1 and Mouse #2 at 14 days post induction. FIG. 10C shows MRI images on the hematoma size of these two mice. FIG. 10D shows better recovery on the striatum in the treatment side. The MRI showed comparable hematoma on both sides at 3 dps, while at 14 days after applying treatment on the right side, we can observe a smaller injury core and a smaller ventricle. This suggests the treatment can relieve the shrinkage of striatum after ICH. MRI scans were obtained at 3 dps.

FIG. 11 is a diagram showing the processes involved in ICH.

### DETAILED DESCRIPTION

This document provides methods and materials involved in treating mammals having had a hemorrhagic stroke. For example, this document provides methods and materials for administering a composition containing exogenous nucleic acid encoding a NeuroD1 polypeptide and nucleic acid encoding a Dlx2 polypeptide to a mammal identified as having had a hemorrhagic stroke.

Any appropriate mammal can be identified as having had a hemorrhagic stroke. For example, humans and other primates such as monkeys can be identified as having had a hemorrhagic stroke.

Any appropriate type of hemorrhagic stroke (e.g., intracranial hemorrhage) can be treated as described herein. For example, intra-axial (within the brain) hemorrhagic strokes such as intracerebral hemorrhages can be treated as described herein. In some cases, extra-axial (outside the brain) hemorrhages such as epidural hemorrhage (e.g., caused by trauma), subdural hemorrhage (e.g., caused by trauma), or subarachnoid hemorrhage (e.g., caused by trauma or aneurysms) can be treated as described herein. About 10-20 percent of all strokes can involve an intracerebral hemorrhage, which can have a high mortality rate of 40 percent within one month and of 54 percent within one year. Causes of intracerebral hemorrhage include hypertension and secondary effects of other diseases such as amyloid angiopathy (e.g., Alzheimer's Disease) or brain tumors. A common location for an intracerebral hemorrhage in the striatum (e.g., about 50 percent). Three models of intracerebral hemorrhage are autologous blood (or lysed blood cell) injection, striatal balloon inflation, and collagenase injection. For autologous blood (or lysed blood cell) injection, about 50-100 μL of whole blood, lysed RBCs, or RBCs plus cellular fraction is injected into the striatum. The hallmark is blood-derived toxicity with no lesion expansion. For striatal balloon inflation, an embolization balloon is inserted into the striatum and slowly inflated with saline. The balloon can be left in place or withdrawn for desired mimic. The hallmark is isolated mechanical effects of mass hematoma. For collagenase injection, about 0.075 Units to 0.4 Units of bacterial collagenase is injected into the striatum to induce basal lamina degradation and ICH. The hallmark is expansive hematoma resulting from in situ rupture, which best mimics ICH in humans.

Intracerebral hemorrhage can bring primary and secondary injuries to the brain. For example, intracerebral hemorrhage can bring primary injury caused by physical pressure induced by hematoma and can bring secondary injury caused by toxicity from blood components, ferroptosis induced by ferric iron (Fe³⁺), and subsequent oxidative stress and inflammation. The methods and materials provided herein (e.g., the administration of nucleic acid encoding a NeuroD1 polypeptide (or a biologically active fragment thereof) and nucleic acid encoding a Dlx2 polypeptide (or a biologically active fragment thereof)) can be used to reduce the severity of one or more primary or secondary injuries to the brain of a mammal (e.g., a human) having had an intracerebral hemorrhage.

In some cases, the hemorrhagic stroke is due to a condition selected from the group consisting of blood vessel rupture, hypertension, aneurysm, ischemic stroke, physical injury, tumor, inflammation, infection, global ischemia, hypoxic-ischemic encephalopathy, meningitis, and dehydration.

In some cases, the hemorrhagic stroke is due to a condition selected from the group consisting of bleeding in the brain, aneurysm, intracranial hematoma, subarachnoid hemorrhage, brain trauma, high blood pressure, weak blood vessels, malformation of blood vessels, ischemic stroke, physical injury, tumor, inflammation, infection; global ischemia, hypoxic-ischemic encephalopathy, meningitis, and dehydration.

In some cases, global ischemia is caused by cardiac arrest or severe hypotension (shock). In some cases, hypoxic-ischemic encephalopathy is caused by hypoxia, hypoglycemia, or anemia.

In some cases, hemorrhagic stroke is due to bleeding in the brain. In some cases, hemorrhagic stroke is due to aneurysm. In some cases, hemorrhagic stroke is due to intracranial hematoma. In some cases, hemorrhagic stroke is due to subarachnoid hemorrhage. In some cases, hemorrhagic stroke is due to brain trauma. In some cases, hemorrhagic stroke is due to high blood pressure. In some cases, hemorrhagic stroke is due to weak blood vessels. In some cases, hemorrhagic stroke is due to malformation of blood vessels. In some cases, hemorrhagic stroke is due to ischemic stroke. In some cases, hemorrhagic stroke is due to physical injury. In some cases, hemorrhagic stroke is due to a tumor. In some cases, hemorrhagic stroke is due to inflammation. In some cases, hemorrhagic stroke is due to infection. In some cases, hemorrhagic stroke is due to global ischemia. In some cases, hemorrhagic stroke is due to hypoxic-ischemic encephalopathy. In some cases, hemorrhagic stroke is due to meningitis. In some cases, hemorrhagic stroke is due to dehydration.

In some cases, administration of a therapeutically effective amount of exogenous nucleic acid encoding a NeuroD1 polypeptide (or a biologically active fragment thereof) and nucleic acid encoding a Dlx2 polypeptide (or a biologically active fragment thereof) to a subject affected by a hemorrhagic stroke mediates: the generation of new glutamatergic neurons by conversion of reactive astrocytes to glutamatergic neurons; reduction of the number of reactive astrocytes; survival of injured neurons including GABAergic and glutamatergic neurons; the generation of new non-reactive astrocytes; the reduction of reactivity of non-converted reactive astrocytes; and reintegration of blood vessels into the injured region.

In some cases, a method or composition provided herein generates new glutamatergic neurons, increasing the number of glutamatergic neurons from a baseline level by between about 1% and 500% after administration of a composition provided herein. In some cases, a method or composition provided herein generates new glutamatergic neurons, increasing the number of glutamatergic neurons from a baseline level by between about 1% and 50%, between about 1% and 100%, between about 1% and 150%, between about 50% and 100%, between about 50% and 150%, between about 50% and 200%, between about 100% and 150%, between about 100% and 200%, between 100% and 250%, between about 150% and 200%, between about 150% and 250%, between about 150% and 300%, between 200% and 250%, between 200% and 300%, between 200% and 350%, between 250% and 300%, between 250% and 350%, between about 250% and 400%, between about 300% and 350%, between about 300% and 400%, between about 300% and 450%, between about 350% and 400%, between about 350% and 450%, between about 350% and 500%, between about 400% and 450%, between about 400% and 500%, or between about 450% and 500% after administration of a composition provided herein.

In some cases, a method or composition provided herein reduces the number of reactive astrocytes by between about 1% and 100% after administration of a composition provided herein. In some cases, a method or composition provided herein reduces the number of reactive astrocytes by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, a method or composition provided herein increases survival of GABAergic neurons by between about 1% and 100% after administration of a composition provided herein compared with no administration. In some cases, a method or composition provided herein increases survival of GABAergic neurons by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein compared with no administration. Any appropriate method can be used to assess increases in survival of GABAergic neurons. For example, immunostaining for 7-aminobutyric acid (GABA), GABA synthesizing enzyme glutamate decarboxylase 67 (GAD67), and/or parvalbumin (PV) can be performed to measure the number of GABAergic neurons. A decrease in the number of GABAergic neurons can indicate GABAergic neuronal loss. When the number remains unchanged, it can indicate that GABAergic neurons survive. An increase in the number of GABAergic neurons can indicate that occurrence of GABAergic regeneration.

In some cases, a method or composition provided herein increases survival of glutamatergic neurons by between about 1% and 100% after administration of a composition provided herein compared with no administration. In some cases, a method or composition provided herein increases survival of glutamatergic neurons by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein compared with no administration. Any appropriate method can be used to assess increases in survival of glutamatergic neurons. For example, immunostaining using markers for glutamatergic neurons can be performed to measure the number of glutamatergic neurons. A decrease in the number of glutamatergic neurons can indicate glutamatergic neuronal loss. When the number remains unchanged, it can indicate that glutamatergic neurons survive. An increase in the number of glutamatergic neurons can indicate the occurrence of glutamatergic regeneration.

In some cases, a method or composition provided herein generates new non-reactive astrocytes, increasing the number of new non-reactive astrocytes from a baseline level by between about 1% and 100% after administration of a composition provided herein. In some cases, a method or composition provided herein generates new non-reactive astrocytes, increasing the number of new non-reactive astrocytes from a baseline level by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In some cases, a method or composition provided herein reduces the reactivity of non-converted reactive astrocytes from a baseline level by between about 1% and 100% after administration of a composition provided herein. In some cases, a method or composition provided here in reduces the reactivity of non-converted reactive astrocytes from a baseline level by between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100% after administration of a composition provided herein.

In some cases, administration of a therapeutically effective amount of exogenous nucleic acid encoding a NeuroD1 polypeptide (or a biologically active fragment thereof) and nucleic acid encoding a Dlx2 polypeptide (or a biologically active fragment thereof) to a subject affected by hemorrhagic stroke mediates: reduced inflammation at the injury site; reduced neuroinhibition at the injury site; re-establishment of normal microglial morphology at the injury site; re-establishment of neural circuits at the injury site, increased blood vessels at the injury site; re-establishment of blood-brain-barrier at the injury site; re-establishment of normal tissue structure at the injury site; and improvement of motor deficits due to the disruption of normal blood flow.

In some cases, administration of a therapeutically effective amount of exogenous nucleic acid encoding a NeuroD1 polypeptide (or a biologically active fragment thereof) and nucleic acid encoding a Dlx2 polypeptide (or a biologically active fragment thereof) to ameliorate the effects of an ICH in an individual subject in need thereof has greater beneficial effects when administered to reactive astrocytes than to quiescent astrocytes.

Treatment with exogenous nucleic acid encoding a NeuroD1 polypeptide (or a biologically active fragment thereof) and nucleic acid encoding a Dlx2 polypeptide (or a biologically active fragment thereof) can be administered to the region of injury as diagnosed by magnetic resonance imaging (MRI). Electrophysiology can assess functional changes in neural firing as caused by neural cell death or injury. Non-invasive methods to assay neural damage include EEG. Disruption of blood flow to a point of injury may be non-invasively assayed via Near Infrared Spectroscopy and fMRI. Blood flow within the region may either be increased, as seen in aneurysms, or decreased, as seen in ischemia. Injury to the CNS caused by disruption of blood flow additionally causes short-term and long-term changes to tissue structure that can be used to diagnose point of injury. In the short term, injury will cause localized swelling. In the long term, cell death will cause points of tissue loss. Non-invasive methods to assay structural changes caused by tissue death include MRI, position emission tomography (PET) scan, computerized axial tomography (CAT) scan, or ultrasound. These methods may be used singularly or in any combination to pinpoint the focus of injury.

As described above, non-invasive methods to assay structural changes caused by tissue death include MRI, CAT scan, or ultrasound. Functional assay may include EEG recording.

In some embodiments of the methods for treating a mammal having had a hemorrhagic stroke as described herein, exogenous NeuroD1 polypeptide (or a biologically active fragment thereof) and Dlx2 polypeptide (or a biologically active fragment thereof) are administered as an expression vector containing a nucleic acid sequence encoding NeuroD1 and Dlx2.

In some embodiments of the methods for treating a neurological disorder as described herein, a viral vector (e.g., an AAV) including a nucleic acid encoding a NeuroD1 polypeptide and a Dlx2 polypeptide is delivered by injection into the brain of a subject, such as stereotaxic intracranial injection or retro-orbital injection. In some cases, the composition containing the adeno-associated virus encoding a NeuroD1 polypeptide and a Dlx2 polypeptide is administered to the brain using two more intracranial injections at the same location in the brain. In some cases, the composition containing the adeno-associated virus encoding a NeuroD1 polypeptide and a Dlx2 polypeptide is administered to the brain using two more intracranial injections at two or more different locations in the brain. In some cases, the composition containing the adeno-associated virus encoding a NeuroD1 polypeptide and a Dlx2 polypeptide is administered to the brain using an one or more extracranial injections.

The term “expression vector” refers to a recombinant vehicle for introducing a nucleic acid encoding a NeuroD1 polypeptide or a biologically active fragment thereof and a nucleic acid encoding a Dlx2 polypeptide or a biologically active fragment thereof into a host cell in vitro or in vivo where the nucleic acid is expressed to produce a NeuroD1 polypeptide and a Dlx2 polypeptide. In particular embodiments, an expression vector including SEQ ID NO: 1 or 3 or a substantially identical nucleic acid sequence is expressed to produce NeuroD1 in cells containing the expression vector. In particular embodiments, an expression vector including SEQ ID NO: 10 or 12 or a substantially identical nucleic acid sequence is expressed to produce Dlx2 in cells containing the expression vector.

The term “recombinant” is used to indicate a nucleic acid construct in which two or more nucleic acids are linked and which are not found linked in nature. Expression vectors include, but are not limited to plasmids, viruses, BACs and YACs. Particular viral expression vectors illustratively include those derived from adenovirus, adeno-associated virus, retrovirus, and lentivirus.

This document provides material and methods for treating the symptoms of a hemorrhagic stroke in a subject in need thereof according to the methods described which include providing a viral vector comprising a nucleic acid encoding NeuroD1 and Dlx2; and delivering the viral vector to the brain of the subject, whereby the viral vector infects glial cells of the central nervous system, respectively, producing infected glial cells and whereby exogenous nucleic acid encoding a NeuroD1 polypeptide or a biologically active fragment thereof and a nucleic acid encoding a Dlx2 polypeptide or a biologically active fragment thereof is expressed in the infected glial cells at a therapeutically effective level, wherein the expression of a NeuroD1 polypeptide and a Dlx2 polypeptide in the infected cells results in a greater number of neurons in the subject compared to an untreated subject having the same neurological condition, whereby the neurological disorder is treated. In addition to the generation of new neurons, the number of reactive glial cells will also be reduced, resulting in less neuroinhibitory factors released, less neuroinflammation, and/or more blood vessels that are also evenly distributed, thereby making local environment more permissive to neuronal growth or axon penetration, hence alleviating neurological conditions.

In some cases, adeno-associated vectors can be used in a method described herein and will infect both dividing and non-dividing cells, at an injection site. Adeno-associated viruses (AAV) are ubiquitous, noncytopathic, replication-incompetent members of ssDNA animal virus of parvoviridae family. Any of various recombinant adeno-associated viruses, such as serotypes 1-9, can be used as described herein. In some cases, an AAV-PHP.eb is used to administer the exogenous NeuroD1 and Dlx2.

A “FLEX” switch approach is used to express NeuroD1 and Dlx2 in infected cells according to some aspects described herein. The terms “FLEX” and “flip-excision” are used interchangeably to indicate a method in which two pairs of heterotypic, antiparallel loxP-type recombination sites are disposed on either side of an inverted NeuroD1 or Dlx2 coding sequence which first undergo an inversion of the coding sequence followed by excision of two sites, leading to one of each orthogonal recombination site oppositely oriented and incapable of further recombination, achieving stable inversion, see for example Schnutgen et al., Nature Biotechnology, 21:562-565 (2003); and Atasoy et al, J. Neurosci., 28:7025-7030 (2008). Since the site-specific recombinase under control of a glial cell-specific promoter will be strongly expressed in glial cells, including reactive astrocytes, NeuroD1 and Dlx2 will also be expressed in glial cells, including reactive astrocytes. Then, when the stop codon in front of NeuroD1 or Dlx2 is removed from recombination, the constitutive or neuron-specific promoter will drive high expression of NeuroD1 and Dlx2, allowing reactive astrocytes to be converted into functional neurons.

According to particular aspects, exogenous nucleic acid encoding a NeuroD1 polypeptide or a biologically active fragment thereof and a nucleic acid encoding a Dlx2 polypeptide or a biologically active fragment thereof are administered to a subject in need thereof by administration of (1) an adeno-associated virus expression vector including a DNA sequence encoding a site-specific recombinase under transcriptional control of an astrocyte-specific promoter such as GFAP or S100b or Aldh1L1; and (2) an adeno-associated virus expression vector including a DNA sequence encoding a NeuroD1 polypeptide and a Dlx2 polypeptide under transcriptional control of a ubiquitous (constitutive) promoter or a neuron-specific promoter wherein the DNA sequence encoding NeuroD1 and Dlx2 is inverted and in the wrong orientation for expression of NeuroD1 and Dlx2 until the site-specific recombinase inverts the inverted DNA sequence encoding NeuroD1 and Dlx2, thereby allowing expression of NeuroD1 and Dlx2.

Site-specific recombinases and their recognition sites include, for example, Cre recombinase along with recognition sites loxP and lox2272 sites, or FLP-FRT recombination, or their combinations.

A composition including an exogenous nucleic acid encoding a NeuroD1 polypeptide or a biologically active fragment thereof and a nucleic acid encoding a Dlx2 polypeptide or a biologically active fragment thereof (e.g., an AAV encoding a NeuroD1 polypeptide and a Dlx2 polypeptide) can be formulated into a pharmaceutical composition for administration into a mammal. For example, a therapeutically effective amount of the composition including an exogenous nucleic acid encoding a NeuroD1 polypeptide or a biologically active fragment thereof and exogenous a nucleic acid encoding a Dlx2 polypeptide or a biologically active fragment thereof can be formulated with one or more pharmaceutically acceptable carriers (additives) and/or diluents. A pharmaceutical composition including an exogenous nucleic acid encoding a NeuroD1 polypeptide or a biologically active fragment thereof and exogenous a nucleic acid encoding a Dlx2 polypeptide or a biologically active fragment thereof (e.g., an AAV encoding a NeuroD1 polypeptide and a Dlx2 polypeptide) can be formulated for various routes of administration, for example, for oral administration as a capsule, a liquid, or the like. In some cases, a viral vector (e.g., AAV) having an exogenous nucleic acid encoding a NeuroD1 polypeptide or a biologically active fragment thereof and exogenous a nucleic acid encoding a Dlx2 polypeptide or a biologically active fragment thereof is administered parenterally, preferably by intravenous injection or intravenous infusion. The administration can be, for example, by intravenous infusion, for example, for 60 minutes, for 30 minutes, or for 15 minutes. In some cases, the intravenous infusion can be between 1 minute and 60 minutes. In some cases, the intravenous infusion can be between 1 minute and 5 minutes, between 1 minute and 10 minutes, between 1 minute and 15 minutes, between 5 minutes and 10 minutes, between 5 minutes and 15 minutes, between 5 minutes and 20 minutes, between 10 minutes and 15 minutes, between 10 minutes and 20 minutes, between 10 minutes and 25 minutes, between 15 minutes and 20 minutes, between 15 minutes and 25 minutes, between 15 minutes and 30 minutes, between 20 minutes and 25 minutes, between 20 minutes and 30 minutes, between 20 minutes and 35 minutes, between 25 minutes and 30 minutes, between 25 minutes and 35 minutes, between 25 minutes and 40 minutes, between 30 minutes and 35 minutes, between 30 minutes and 40 minutes, between 30 minutes and 45 minutes, between 35 minutes and 40 minutes, between 35 minutes and 45 minutes, between 35 minutes and 50 minutes, between 40 minutes and 45 minutes, between 40 minutes and 50 minutes, between 40 minutes and 55 minutes, between 45 minutes and 50 minutes, between 45 minutes and 55 minutes, between 45 minutes and 60 minutes, between 50 minutes and 55 minutes, between 50 minutes and 60 minutes, or between 55 minutes and 60 minutes.

In some cases, administration can be provided to a mammal between 1 day and 60 days post hemorrhagic stroke. In some cases, administration can be provided to a mammal between 1 day and 5 days, between 1 day and 10 days, between 1 day and 15 days, between 5 days and 10 days, between 5 days and 15 days, between 5 days and 20 days, between 10 days and 15 days, between 10 days and 20 days, between 10 days and 25 days, between 15 days and 20 days, between 15 days and 25 days, between 15 days and 30 days, between 20 days and 25 days, between 20 days and 30 days, between 20 days and 35 days, between 25 days and 30 days, between 25 days and 35 days, between 25 days and 40 days, between 30 days and 35 days, between 30 days and 40 days, between 30 days and 45 days, between 35 days and 40 days, between 35 days and 45 days, between 35 days and 50 days, between 40 days and 45 days, between 40 days and 50 days, between 40 days and 55 days, between 45 days and 50 days, between 45 days and 55 days, between 45 days and 60 days, between 50 days and 55 days, between 50 days and 60 days, or between 55 days and 60 days post hemorrhagic stroke.

In some cases, administration can be provided to a mammal at the time of a hemorrhagic stroke. In some cases, administration can be provided to a mammal 1 day post hemorrhagic stroke. In some cases, administration can be provided to a mammal 2 days post hemorrhagic stroke. In some cases, administration can be provided to a mammal 3 days post hemorrhagic stroke. In some cases, administration can be provided to a mammal 4 days post hemorrhagic stroke. In some cases, administration can be provided to a mammal 5 days post hemorrhagic stroke. In some cases, administration can be provided to a mammal 6 day post hemorrhagic stroke. In some cases, administration can be provided to a mammal 7 days post hemorrhagic stroke. In some cases, administration can be provided to a mammal 1 week post hemorrhagic stroke. In some cases, administration can be provided to a mammal 2 weeks post hemorrhagic stroke. In some cases, administration can be provided to a mammal 3 weeks post hemorrhagic stroke. In some cases, administration can be provided to a mammal 4 weeks post hemorrhagic stroke. In some cases, administration can be provided to a mammal 5 weeks post hemorrhagic stroke. In some cases, administration can be provided to a mammal 6 weeks post hemorrhagic stroke. In some cases, administration can be provided to a mammal 7 weeks post hemorrhagic stroke. In some cases, administration can be provided to a mammal 8 weeks post hemorrhagic stroke.

In some cases, the viral vector (e.g., AAV encoding a NeuroD1 polypeptide and Dlx2 polypeptide) is administered locally by injection to the brain during a surgery. Compositions which are suitable for administration by injection and/or infusion include solutions and dispersions, and powders from which corresponding solutions and dispersions can be prepared. Such compositions will comprise the viral vector and at least one suitable pharmaceutically acceptable carrier. Suitable pharmaceutically acceptable carriers for intravenous administration include, but not limited to, bacterostatic water, Ringer's solution, physiological saline, phosphate buffered saline (PBS), and CREMOPHOR EL® (polyethoxylated castor oil). Sterile compositions for the injection and/or infusion can be prepared by introducing the viral vector (e.g., AAV encoding a NeuroD1 polypeptide and a Dlx2 polypeptide) in the required amount into an appropriate carrier, and then sterilizing by filtration. Compositions for administration by injection or infusion should remain stable under storage conditions after their preparation over an extended period of time. The compositions can contain a preservative for this purpose. Suitable preservatives include chlorobutanol, phenol, ascorbic acid, and thimerosal.

In some embodiments, the gene delivery vector can be an AAV vector. For example, an AAV vector can be selected from the group of: an AAV2 vector, an AAV5 vector, an AAV8 vector, an AAV1 vector, an AAV7 vector, an AAV9 vector, an AAV3 vector, an AAV6 vector, an AAV10 vector, and an AAV11 vector.

A pharmaceutical composition can be formulated for administration in solid or liquid form including, without limitation, sterile solutions, suspensions, sustained-release formulations, tablets, capsules, pills, powders, and granules. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

Additional pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the term “adeno-associated virus particle” refers to packaged capsid forms of the AAV virus that transmits its nucleic acid genome to cells.

An effective amount of composition containing an exogenous NeuroD1 and Dlx2 can be any amount that ameliorates the symptoms of the neurological disorder within a mammal (e.g., a human) without producing severe toxicity to the mammal. For example, an effective amount of adeno-associated virus encoding a NeuroD1 polypeptide and a Dlx2 polypeptide can be a concentration from about 10¹⁰ to 10¹⁴ adeno-associated virus particles/mL. If a particular mammal fails to respond to a particular amount, then the amount of the AAV encoding a NeuroD1 polypeptide and a Dlx2 polypeptide can be increased. In some cases, an effective amount of adeno-associated virus encoding a NeuroD1 and a Dlx2 polypeptide can be between 10¹⁰ adeno-associated virus particles/mL and 10¹¹ adeno-associated virus particles/mL, between 10¹⁰ adeno-associated virus particles/mL and 10¹² adeno-associated virus particles/mL, between 10¹⁰ adeno-associated virus particles/mL and 10¹⁰ adeno-associated virus particles/mL, between 10¹¹ adeno-associated virus particles/mL and 10¹² adeno-associated virus particles/mL, between 10¹¹ adeno-associated virus particles/mL and 10¹³ adeno-associated virus particles/mL, between 10¹¹ adeno-associated virus particles/mL and 10¹⁴ adeno-associated virus particles/mL, between 10¹² adeno-associated virus particles/mL and 10¹³ adeno-associated virus particles/mL, between 10¹² adeno-associated virus particles/mL and 10¹⁴ adeno-associated virus particles/mL, or between 10¹³ adeno-associated virus particles/mL and 10¹⁴ adeno-associated virus particles/mL. Factors that are relevant to the amount of viral vector (e.g., an AAV encoding a NeuroD1 polypeptide and a Dlx2 polypeptide) to be administered are, for example, the route of administration of the viral vector, the nature and severity of the disease, the disease history of the patient being treated, and the age, weight, height, and health of the patient to be treated. In some cases, the expression level of the transgene, which is required to achieve a therapeutic effect, the immune response of the patient, as well as the stability of the gene product are relevant for the amount to be administered. In some cases, the administration of the viral vector (e.g., an AAV encoding an exogenous NeuroD1 and Dlx2) occurs in an amount which leads to a complete or substantially complete healing of the dysfunction or disease of the brain.

In some cases, an effective amount of composition containing an exogenous NeuroD1 and Dlx2 can be any administered at a controlled flow rate of about 0.1 μL/minute to about 5 μL/minute.

In some cases, the controlled flow rate is between 0.1 μL/minute and 0.2 μL/minute, between 0.1 μL/minute and 0.3 μL/minute, between 0.1 μL/minute and 0.4 μL/minute, between 0.2 μL/minute and 0.3 μL/minute, between 0.2 μL/minute and 0.4 μL/minute, between 0.2 μL/minute and 0.5 μL/minute, between 0.3 μL/minute and 0.4 μL/minute, between 0.3 μL/minute and 0.5 μL/minute, between 0.3 μL/minute and 0.6 μL/minute, between 0.4 μL/minute and 0.5 μL/minute, between 0.4 μL/minute and 0.6 μL/minute, between 0.4 μL/minute and 0.7 μL/minute, between 0.5 μL/minute and 0.6 μL/minute, between 0.5 μL/minute and 0.7 μL/minute, between 0.5 μL/minute and 0.8 μL/minute, between 0.6 μL/minute and 0.7 μL/minute, between 0.6 μL/minute and 0.8 μL/minute, between 0.6 μL/minute and 0.9 μL/minute, between 0.7 μL/minute and 0.8 μL/minute, between 0.7 μL/minute and 0.9 μL/minute, between 0.7 μL/minute and 1.0 μL/minute, between 0.8 μL/minute and 0.9 μL/minute, between 0.8 μL/minute and 1.0 μL/minute, between 0.8 μL/minute and 1.1 μL/minute, between 0.9 μL/minute and 1.0 μL/minute, between 0.9 μL/minute and 1.1 μL/minute, between 0.9 μL/minute and 1.2 μL/minute, between 1.0 μL/minute and 1.1 μL/minute, between 1.0 μL/minute and 1.2 μL/minute, between 1.0 μL/minute and 1.3 μL/minute, between 1.1 μL/minute and 1.2 μL/minute, between 1.1 μL/minute and 1.3 μL/minute, between 1.1 μL/minute and 1.4 μL/minute, between 1.2 μL/minute and 1.3 μL/minute, between 1.2 μL/minute and 1.4 μL/minute, between 1.2 μL/minute and 1.5 μL/minute, between 1.3 μL/minute and 1.4 μL/minute, between 1.3 μL/minute and 1.5 μL/minute, between 1.3 μL/minute and 1.6 μL/minute, between 1.4 μL/minute and 1.5 μL/minute, between 1.4 μL/minute and 1.6 μL/minute, between 1.4 μL/minute and 1.7 μL/minute, between 1.5 μL/minute and 1.6 μL/minute, between 1.5 μL/minute and 1.7 μL/minute, between 1.5 μL/minute and 1.8 μL/minute, between 1.6 μL/minute and 1.7 μL/minute, between 1.6 μL/minute and 1.8 μL/minute, between 1.6 μL/minute and 1.9 μL/minute, between 1.7 μL/minute and 1.8 μL/minute, between 1.7 μL/minute and 1.9 μL/minute, between 1.7 μL/minute and 2.0 μL/minute, between 1.8 μL/minute and 1.9 μL/minute, between 1.8 μL/minute and 2.0 μL/minute, between 1.8 μL/minute and 2.1 μL/minute, between 1.9 μL/minute and 2.0 μL/minute, between 1.9 μL/minute and 2.1 μL/minute, between 1.9 μL/minute and 2.2 μL/minute, between 2.0 μL/minute and 2.1 μL/minute, between 2.0 μL/minute and 2.2 μL/minute, between 2.0 μL/minute and 2.3 μL/minute, between 2.1 μL/minute and 2.2 μL/minute, between 2.1 μL/minute and 2.3 μL/minute, between 2.1 μL/minute and 2.4 μL/minute, between 2.2 μL/minute and 2.3 μL/minute, between 2.2 μL/minute and 2.4 μL/minute, between 2.2 μL/minute and 2.5 μL/minute, between 2.3 μL/minute and 2.4 μL/minute, between 2.3 μL/minute and 2.5 μL/minute, between 2.3 μL/minute and 2.6 μL/minute, between 2.4 μL/minute and 2.5 μL/minute, between 2.4 μL/minute and 2.6 μL/minute, between 2.4 μL/minute and 2.7 μL/minute, between 2.5 μL/minute and 2.6 μL/minute, between 2.5 μL/minute and 2.7 μL/minute, between 2.5 μL/minute and 2.8 μL/minute, between 2.6 μL/minute and 2.7 μL/minute, between 2.6 μL/minute and 2.8 μL/minute, between 2.6 μL/minute and 2.9 μL/minute, between 2.7 μL/minute and 2.8 μL/minute, between 2.7 μL/minute and 2.9 μL/minute, between 2.7 μL/minute and 3.0 μL/minute, between 2.8 μL/minute and 2.9 μL/minute, between 2.8 μL/minute and 3.0 μL/minute, between 2.8 μL/minute and 3.1 μL/minute, between 2.9 μL/minute and 3.0 μL/minute, between 2.9 μL/minute and 3.1 μL/minute, between 2.9 μL/minute and 3.2 μL/minute, between 3.0 μL/minute and 3.1 μL/minute, between 3.0 μL/minute and 3.2 μL/minute, between 3.0 μL/minute and 3.3 μL/minute, between 3.1 μL/minute and 3.2 μL/minute, between 3.1 μL/minute and 3.3 μL/minute, between 3.1 μL/minute and 3.4 μL/minute, between 3.2 μL/minute and 3.3 μL/minute, between 3.2 μL/minute and 3.4 μL/minute, between 3.2 μL/minute and 3.5 μL/minute, between 3.3 μL/minute and 3.4 μL/minute, between 3.3 μL/minute and 3.5 μL/minute, between 3.3 μL/minute and 3.6 μL/minute, between 3.4 μL/minute and 3.5 μL/minute, between 3.4 μL/minute and 3.6 μL/minute, between 3.4 μL/minute and 3.7 μL/minute, between 3.5 μL/minute and 3.6 μL/minute, between 3.5 μL/minute and 3.7 μL/minute, between 3.5 μL/minute and 3.8 μL/minute, between 3.6 μL/minute and 3.7 μL/minute, between 3.6 μL/minute and 3.8 μL/minute, between 3.6 μL/minute and 3.9 μL/minute, between 3.7 μL/minute and 3.8 μL/minute, between 3.7 μL/minute and 3.9 μL/minute, between 3.7 μL/minute and 4.0 μL/minute, between 3.8 μL/minute and 3.9 μL/minute, between 3.8 μL/minute and 4.0 μL/minute, between 3.8 μL/minute and 4.1 μL/minute, between 3.9 μL/minute and 4.0 μL/minute, between 3.9 μL/minute and 4.1 μL/minute, between 3.9 μL/minute and 4.2 μL/minute, between 4.0 μL/minute and 4.1 μL/minute, between 4.0 μL/minute and 4.2 μL/minute, between 4.0 μL/minute and 4.3 μL/minute, between 4.1 μL/minute and 4.2 μL/minute, between 4.1 μL/minute and 4.3 μL/minute, between 4.1 μL/minute and 4.4 μL/minute, between 4.2 μL/minute and 4.3 μL/minute, between 4.2 μL/minute and 4.4 μL/minute, between 4.2 μL/minute and 4.5 μL/minute, between 4.3 μL/minute and 4.4 μL/minute, between 4.3 μL/minute and 4.5 μL/minute, between 4.3 μL/minute and 4.6 μL/minute, between 4.4 μL/minute and 4.5 μL/minute, between 4.4 μL/minute and 4.6 μL/minute, between 4.4 μL/minute and 4.7 μL/minute, between 4.5 μL/minute and 4.6 μL/minute, between 4.5 μL/minute and 4.7 μL/minute, between 4.5 μL/minute and 4.8 μL/minute, between 4.6 μL/minute and 4.7 μL/minute, between 4.6 μL/minute and 4.8 μL/minute, between 4.6 μL/minute and 4.9 μL/minute, between 4.7 μL/minute and 4.8 μL/minute, between 4.7 μL/minute and 4.9 μL/minute, between 4.7 μL/minute and 5.0 μL/minute, 4.8 μL/minute and 4.9 μL/minute, between 4.8 μL/minute and 5.0 μL/minute, or between 4.9 μL/minute and 5.0 μL/minute.

The viral vector (e.g., an AAV containing a nucleic acid encoding for a NeuroD1 polypeptide and a nucleic acid encoding for a Dlx2 polypeptide) can be administered in an amount corresponding to a dose of virus in the range of about 1.0×10¹⁰ to about 1.0×10¹⁴ vg/kg (virus genomes per kg body weight). In some cases, the viral vector (e.g., an AAV containing a nucleic acid encoding for a NeuroD1 polypeptide and a nucleic acid encoding for a Dlx2 polypeptide) can be administered in amount corresponding to a dose of virus in the range of about 1.0×10¹¹ to about 1.0×10¹² vg/kg, a range of about 5.0×10¹¹ to about 5.0×10¹² vg/kg, or a range of about 1.0×10¹² to about 5.0×10¹¹ is still more preferred. In some cases, the viral vector (e.g., an AAV containing a nucleic acid encoding for a NeuroD1 polypeptide and a nucleic acid encoding for a Dlx2 polypeptide) is administered in an amount corresponding to a dose of about 2.5×10¹² vg/kg. In some cases, the effective amount of the viral vector (e.g., an AAV containing a nucleic acid encoding for a NeuroD1 polypeptide and a nucleic acid encoding for a Dlx2 polypeptide) can be a volume of about 1 μL to about 500 μL, corresponding to the volume for the vg/kg (virus genomes per kg body weight) doses described herein. In some cases, the amount of the viral vector to be administered (e.g., an AAV containing a nucleic acid encoding for a NeuroD1 polypeptide and a nucleic acid encoding for a Dlx2 polypeptide) is adjusted according to the strength of the expression of one or more exogenous nucleic acids encoding a polypeptide (e.g., NeuroD1 and Dlx2).

In some cases, the effective volume administered of the viral vector is between 1 μL and 25 μL, between 1 μL and 50 μL, between 1 μL and 75 μL, between 25 μL and 50 μL, between 25 μL and 75 μL, between 25 μL and 100 μL, between 50 μL and 75 μL, between 50 L and 100 μL, between 50 μL and 125 μL, between 75 μL and 100 μL, between 75 μL and 125 μL, between 75 μL and 150 μL, between 100 μL and 125 μL, between 100 μL and 150 μL, between 100 μL and 175 μL, between 125 μL and 150 μL, between 125 μL and 175 μL, between 125 μL and 200 μL, between 150 μL and 175 μL, between 150 μL and 200 μL, between 150 μL and 225 μL, between 175 μL and 200 μL, between 175 μL and 225 μL, between 175 μL and 250 μL, between 200 μL and 225 μL, between 200 μL and 250 μL, between 200 μL and 275 μL, between 225 μL and 250 μL, between 225 μL and 275 μL, between 225 μL and 300 μL, between 250 μL and 275 μL, between 250 μL and 300 μL, between 250 μL and 325 μL, between 275 μL and 300 μL, between 275 μL and 325 μL, between 275 μL and 350 μL, between 300 μL and 325 μL, between 300 μL and 350 μL, between 300 μL and 375 μL, between 325 μL and 350 μL, between 325 μL and 375 μL, between 325 μL and 400 μL, between 350 μL and 375 μL, between 350 μL and 400 μL, between 350 μL and 425 μL, between 375 μL and 400 μL, between 375 μL and 425 μL, between 375 μL and 450 μL, between 400 μL and 425 μL, between 400 μL and 450 μL, between 400 μL and 475 μL, between 425 μL and 450 μL, between 425 μL and 475 μL, between 425 μL and 500 μL, between 450 μL and 475 μL, between 450 μL and 500 μL, or between 475 μL and 500 μL.

In some cases, an adeno-associated virus vector including a nucleic acid encoding a NeuroD1 polypeptide and a Dlx2 polypeptide under transcriptional control of a ubiquitous (constitutive) promoter or a neuron-specific promoter wherein the nucleic acid sequence encoding NeuroD1 and Dlx2 is inverted and in the wrong orientation for expression of NeuroD1 and Dlx2 and further includes sites for recombinase activity by a site specific recombinase, until the site-specific recombinase inverts the inverted nucleic acid sequence encoding NeuroD1 and Dlx2, thereby allowing expression of NeuroD1 and Dlx2 polypeptides, is delivered by stereotactic injection into the brain of a subject along with an adeno-associated virus encoding a site specific recombinase.

In some cases, an adeno-associated virus vector including a nucleic acid encoding a NeuroD1 polypeptide and a Dlx2 polypeptide under transcriptional control of a ubiquitous (constitutive) promoter or a neuron-specific promoter wherein the nucleic acid sequence encoding a NeuroD1 polypeptide and a Dlx2 polypeptide is inverted and in the wrong orientation for expression of NeuroD1 and Dlx2 and further includes sites for recombinase activity by a site specific recombinase, until the site-specific recombinase inverts the inverted nucleic acid sequence encoding NeuroD1 and Dlx2, thereby allowing expression of a NeuroD1 polypeptide and a Dlx2 polypeptide, is delivered by stereotactic injection into the brain of a subject along with an adeno-associated virus encoding a site specific recombinase in the region of or at the site interest.

In some cases, the site-specific recombinase is Cre recombinase and the sites for recombinase activity are recognition sites loxP and lox2272 sites.

In some cases, treatment of a subject exogenous nucleic acid encoding a NeuroD1 polypeptide and a Dlx2 polypeptide is monitored during or after treatment to monitor progress and/or final outcome of the treatment. Post-treatment success of neuronal cell integration and restoration of tissue microenvironment can be diagnosed by restoration or near-restoration of normal electrophysiology, blood flow, tissue structure, and function. Non-invasive methods to assay neural function include EEG. Blood flow may be non-invasively assayed via Near Infrared Spectroscopy and fMRI. Non-invasive methods to assay tissue structure include MRI, CAT scan, PET scan, or ultrasound. Behavioral assays may be used to non-invasively assay for restoration of brain function. The behavioral assay should be matched to the loss of function caused by original brain injury. For example, if injury caused paralysis, the patient's mobility and limb dexterity should be tested. If injury caused loss or slowing of speech, patient's ability to communicate via spoken word should be assayed. Restoration of normal behavior post treatment with exogenous nucleic acid encoding a NeuroD1 polypeptide and a Dlx2 polypeptide indicates successful creation and integration of effective neuronal circuits. These methods may be used singularly or in any combination to assay for neural function and tissue health. Assays to evaluate treatment may be performed at any point, such as 1 day, 2 days, 3 days, one week, 2 weeks, 3 weeks, one month, two months, three months, six months, one year, or later, after NeuroD1 and Dlx2 treatment. Such assays may be performed prior to NeuroD1 and Dlx2 treatment in order to establish a baseline comparison if desired.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Asubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, P A, 2003; Herdewijn, p. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, Cold Spring Harbor Laboratory Press, 3rd Ed.; Dec. 15, 2002,ISBN-10:0879695919; Kursad Turksen (Ed.), Embryonic Stem Cells: Methods and Protocols in Methods in Molecular Biology, 2002; 185, Human Press: Current Protocols in Stem Cell Biology, ISBN:9780470151808.

As used herein, the singular terms “a,” “an,” and “the” are not intended to be limiting and include plural referents unless explicitly stated otherwise or the context clearly indicates otherwise.

As used herein, the term or “NeuroD1 protein” refers to a bHLH proneural transcription factor involved in embryonic brain development and in adult neurogenesis, see Cho et al., Mol, Neurobiol., 30:35-47 (2004); Kuwabara et al., Nature Neurosci., 12:1097-1105 (2009); and Gao et al., Nature Neurosci., 12:1090-1092 (2009). NeuroD1 is expressed late in development, mainly in the nervous system and is involved in neuronal differentiation, maturation, and survival.

The term “NeuroD1 protein” or “exogenous NeuroD1” encompasses human NeuroD1 protein, identified herein as SEQ ID NO: 2 and mouse NeuroD1 protein, identified herein as SEQ ID NO: 4. In addition to the NeuroD1 protein of SEQ ID NO: 2 and SEQ ID NO: 4, the term “NeuroD1 protein” encompasses variants of NeuroD1 protein, such as variants of SEQ ID NO: 2 and SEQ ID NO: 4, which may be included in a method described herein. As used herein, the term “variant” refers to naturally occurring genetic variations and recombinantly prepared variations, each of which contain one or more changes in its amino acid sequence compared to a reference NeuroD1 protein, such as SEQ ID NO: 2 or SEQ ID NO: 4. Such changes include those in which one or more amino acid residues have been modified by amino acid substitution, addition or deletion. The term “variant” encompasses orthologs of human NeuroD1, including for example mammalian and bird NeuroD1, such as, but not limited to NeuroD1 orthologs from a non-human primate, cat, dog, sheep, goat, horse, cow, pig, bird, poultry animal and rodent such as but not limited to mouse and rat. In a non-limiting example, mouse NeuroD1, exemplified herein as amino acid sequence SEQ ID NO: 4, is an ortholog of human NeuroD1.

In some cases, preferred variants have at least 75%, 80%, 85%, 90%, 91%, 92%, 93%, 94%, 95%, 96%, 97%, 98%, or 99% identity to SEQ ID NO: 2 or SEQ ID NO: 4.

Mutations can be introduced using standard molecular biology techniques, such as site-directed mutagenesis and PCR-mediated mutagenesis. One of skill in the art will recognize that one or more amino acid mutations can be introduced without altering the functional properties of the NeuroD1 protein. For example, one or more amino acid substitutions, additions, or deletions can be made without altering the functional properties of the NeuroD1 protein of SEQ ID NO: 2 or 4.

Conservative amino acid substitutions can be made in a NeuroD1 protein to produce a NeuroD1 protein variant. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate and glutamate; basic amino acids include histidine, lysine, and arginine; aliphatic amino acids include isoleucine, leucine, and valine; aromatic amino acids include phenylalanine, glycine, tyrosine, and tryptophan; polar amino acids include aspartate, glutamate, histidine, lysine, asparagine, glutamine, arginine, serine, threonine, and tyrosine; and hydrophobic amino acids include alanine, cysteine, phenylalanine, glycine, isoleucine, leucine, methionine, proline, valine, and tryptophan; and conservative substitutions include substitution among amino acids within each group. Amino acids may also be described in terms of relative size with alanine, cysteine, aspartate, glycine, asparagine, proline, threonine, serine, and valine, all typically being considered to be small.

NeuroD1 variants can include synthetic amino acid analogs, amino acid derivatives, and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, PNAS, 87:2264-2268 (1990), modified as in Karlin and Altschul, PNAS, 90:5873-5877 (1993). Such an algorithm is incorporated into the BLAST® (Basic Local Alignment Search Tool) nucleotide (also known as blastn) and BLAST® translated nucleotide (also known as blastx) programs of Altschul et al., J. Mol. Biol., 215:403 (1990). BLAST® nucleotide searches are performed with the BLAST® nucleotide program parameters set, e.g., for score=100, wordlength=12 to obtain nucleotide sequences homologous to a nucleic acid molecule described herein.

BLAST® protein searches are performed with the blastx program parameters set, e.g., to score 50, word length=3 to obtain amino acid sequences homologous to a protein molecule described herein. To obtain gapped alignments for comparison purposes, Gapped BLAST® are utilized as described in Altschul et al., Nucleic Acids Res., 25:3389-3402 (1997). Alternatively, PSI (Position-Specific Iterated) BLAST® is used to perform an iterated search which detects distant relationships between molecules. When utilizing BLAST®, Gapped BLAST®, and PSI BLAST® programs, the default parameters of the respective programs (e.g., of blastx and blastn) are used (see, e.g., the NCBI website).

Another preferred, non-limiting example of a mathematical algorithm utilized for the comparison of sequences is the algorithm of Myers and Miller, CABIOS, 4:11-17 (1988). Such an algorithm is incorporated in the ALIGN program (version 2.0), which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

The term “NeuroD1 protein” encompasses fragments of the NeuroD1 protein, such as fragments of SEQ ID NOs. 2 and 4 and variants thereof, operable in a method or composition described herein.

NeuroD1 proteins and nucleic acids may be isolated from natural sources, such as the brain of an organism or cells of a cell line which expresses NeuroD1. Alternatively, NeuroD1 protein or nucleic acid may be generated recombinantly, such as by expression using an expression construct, in vitro or in vivo. NeuroD1 proteins and nucleic acids may also be synthesized by well-known methods.

NeuroD1 included in a method or composition described herein can be produced using recombinant nucleic acid technology. Recombinant NeuroD1 production includes introducing a recombinant expression vector encompassing a DNA sequence encoding NeuroD1 into a host cell.

In some cases, a nucleic acid sequence encoding NeuroD1 introduced into a host cell to produce NeuroD1 encodes SEQ ID NO: 2, SEQ ID NO: 4, or a variant thereof.

In some cases, the nucleic acid sequence identified herein as SEQ ID NO: 1 encodes SEQ ID NO: 2 and is included in an expression vector and expressed to produce NeuroD1. In some cases, the nucleic acid sequence identified herein as SEQ ID NO: 3 encodes SEQ ID NO: 4 and is included in an expression vector and expressed to produce NeuroD1. In some cases, the nucleic acid sequence identified herein as SEQ ID NO: 10 encodes SEQ ID NO: 11 and is included in an expression vector and expressed to produce Dlx2. In some cases, the nucleic acid sequence identified herein as SEQ ID NO: 12 encodes SEQ ID NO: 13 and is included in an expression vector and expressed to produce Dlx2.

It is appreciated that due to the degenerate nature of the genetic code, nucleic acid sequences substantially identical to SEQ ID NOs. 1 and 3 encode NeuroD1 and variants of NeuroD1, and that such alternate nucleic acids may be included in an expression vector and expressed to produce NeuroD1 and variants of NeuroD1. One of skill in the art will appreciate that a fragment of a nucleic acid encoding NeuroD1 protein can be used to produce a fragment of a NeuroD1 protein.

As used herein, the term “Dlx2” refers to distal-less homeobox 2 that acts as a transcriptional activator and plays a role in terminal differentiation of interneurons, such as amacrine and bipolar cells in the developing retina. Dlx2 plays a regulatory role in the development of the ventral forebrain, and may play a role in craniofacial patterning and morphogenesis. The term “Dlx2 protein” or “exogenous Dlx2” encompasses human Dlx2 protein, identified herein as SEQ ID NO: 11 and mouse Dlx2 protein, identified herein as SEQ ID NO: 13. In addition to the Dlx2 protein of SEQ ID NO: 11 and SEQ ID NO: 13, the term “Dlx2 protein” encompasses variants of Dlx2 protein, such as variants of SEQ ID NO: 11 and SEQ ID NO: 13, which may be included in a method described herein.

An expression vector contains a nucleic acid that includes segment encoding a polypeptide of interest operably linked to one or more regulatory elements that provide for transcription of the segment encoding the polypeptide of interest. The term “operably linked” as used herein refers to a nucleic acid in functional relationship with a second nucleic acid. The term “operably linked” encompasses functional connection of two or more nucleic acid molecules, such as a nucleic acid to be transcribed and a regulatory element. The term “regulatory element” as used herein refers to a nucleotide sequence which controls some aspect of the expression of an operably linked nucleic acid. Exemplary regulatory elements include an enhancer, such as, but not limited to: woodchuck hepatitis virus posttranscriptional regulatory element (WPRE); an internal ribosome entry site (IRES) or a 2A domain; an intron; an origin of replication; a polyadenylation signal (pA); a promoter; a transcription termination sequence; and an upstream regulatory domain, which contribute to the replication, transcription, post-transcriptional processing of an operably linked nucleic acid sequence. Those of ordinary skill in the art are capable of selecting and using these and other regulatory elements in an expression vector with no more than routine experimentation.

The term “promoter” as used herein refers to a DNA sequence operably linked to a nucleic acid sequence to be transcribed such as a nucleic acid sequence encoding NeuroD1 and/or a nucleic acid sequence encoding Dlx2. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

As will be recognized by the skilled artisan, the 5′ non-coding region of a gene can be isolated and used in its entirety as a promoter to drive expression of an operably linked nucleic acid. Alternatively, a portion of the 5′ non-coding region can be isolated and used to drive expression of an operably linked nucleic acid. In general, about 500-6000 bp of the 5′ non-coding region of a gene is used to drive expression of the operably linked nucleic acid.

Optionally, a portion of the 5′ non-coding region of a gene containing a minimal amount of the 5′ non-coding region needed to drive expression of the operably linked nucleic acid is used. Assays to determine the ability of a designated portion of the 5′ non-coding region of a gene to drive expression of the operably linked nucleic acid are well-known in the art.

Particular promoters used to drive expression of NeuroD1 and/or Dlx2 according to methods described herein are “ubiquitous” or “constitutive” promoters, that drive expression in many, most, or all cell types of an organism into which the expression vector is transferred. Non-limiting examples of ubiquitous promoters that can be used in expression of NeuroD1 and/or Dlx2 are cytomegalovirus promoter; simian virus 40 (SV40) early promoter; rous sarcoma virus promoter; adenovirus major late promoter; beta actin promoter; glyceraldehyde 3-phosphate dehydrogenase; glucose-regulated protein 78 promoter; glucose-regulated protein 94 promoter; heat shock protein 70 promoter; beta-kinesin promoter; ROSA promoter; ubiquitin B promoter; eukaryotic initiation factor 4A1 promoter and elongation Factor I promoter; all of which are well-known in the art and which can be isolated from primary sources using routine methodology or obtained from commercial sources. Promoters can be derived entirely from a single gene or can be chimeric, having portions derived from more than one gene.

Combinations of regulatory sequences may be included in an expression vector and used to drive expression of NeuroD1 and/or Dlx2. A non-limiting example included in an expression vector to drive expression of NeuroD1 and/or Dlx2 is the CAG promoter which combines the cytomegalovirus CMV early enhancer element and chicken beta-actin promoter.

Particular promoters used to drive expression of NeuroD1 and/or Dlx2 according to methods described herein are those that drive expression preferentially in glial cells, particularly astrocytes and/or NG2 cells. Such promoters are termed “astrocyte-specific” and/or “NG2 cell-specific” promoters.

Non-limiting examples of astrocyte-specific promoters are glial fibrillary acidic protein (GFAP) promoter and aldehyde dehydrogenase 1 family, member L1 (Aldh1L1) promoter. Human GFAP promoter is shown herein as SEQ ID NO:6. Mouse Aldh1L1 promoter is shown herein as SEQ ID NO:7.

A non-limiting example of an NG2 cell-specific promoter is the promoter of the chondroitin sulfate proteoglycan 4 gene, also known as neuron-glial antigen 2 (NG2). Human NG2 promoter is shown herein as SEQ ID NO:8.

Particular promoters used to drive expression of NeuroD1 and/or Dlx2 according to methods described herein are those that drive expression preferentially in reactive glial cells, particularly reactive astrocytes and/or reactive NG2 cells. Such promoters are termed “reactive astrocyte-specific” and/or “reactive NG2 cell-specific” promoters.

A non-limiting example of a “reactive astrocyte-specific” promoter is the promoter of the lipocalin 2 (lcn2) gene. Mouse lcn2 promoter is shown herein as SEQ ID NO:5.

Homologues and variants of ubiquitous and cell type-specific promoters may be used in expressing NeuroD1 and/or Dlx2.

In some cases, promoter homologues and promoter variants can be included in an expression vector for expressing NeuroD1 and/or Dlx2. The terms “promoter homologue” and “promoter variant” refer to a promoter which has substantially similar functional properties to confer the desired type of expression, such as cell type-specific expression of NeuroD1 (and/or Dlx2) or ubiquitous expression of NeuroD1 (and/or Dlx2), on an operably linked nucleic acid encoding NeuroD1 (and/or Dlx2) compared to those disclosed herein. For example, a promoter homologue or variant has substantially similar functional properties to confer cell type-specific expression on an operably linked nucleic acid encoding NeuroD1 (and/or Dlx2) compared to GFAP, S100b, Aldh1L1, NG2, lcn2 and CAG promoters.

One of skill in the art will recognize that one or more nucleic acid mutations can be introduced without altering the functional properties of a given promoter. Mutations can be introduced using standard molecular biology techniques, such as site-directed mutagenesis and PCR-mediated mutagenesis, to produce promoter variants. As used herein, the term “promoter variant” refers to either an isolated naturally occurring or a recombinantly prepared variation of a reference promoter, such as, but not limited to, GFAP, S100b, Aldh1L1, NG2, lcn2, and pCAG promoters.

It is known in the art that promoters from other species are functional, e.g. the mouse AldhlL1 promoter is functional in human cells. Homologues and homologous promoters from other species can be identified using bioinformatics tools known in the art, see for example, Xuan et al., Genome Biol., 6:R72 (2005); Zhao et al., Nucl. Acid Res., 33:D103-107 (2005); and Halees et al., Nucl. Acid Res., 31:3554-3559 (2003).

Structurally, homologues and variants of cell type-specific promoters of NeuroD1 or and/or ubiquitous promoters have at least 80%, 81%, 82%, 83%, 84%, 85%, 86%, 87%, 88%, 89, 90%, 91%, 92%, 93%, 94%, 95%, 96%, 97%, 98%, 99%, or greater, nucleic acid sequence identity to the reference developmentally regulated and/or ubiquitous promoter and include a site for binding of RNA polymerase and, optionally, one or more binding sites for transcription factors.

A nucleic acid sequence which is substantially identical to SEQ ID NO:1 or SEQ ID NO:3 is characterized as having a complementary nucleic acid sequence capable of hybridizing to SEQ ID NO:1 or SEQ ID NO:3 under high stringency hybridization conditions.

In addition to one or more nucleic acids encoding NeuroD1, one or more nucleic acid sequences encoding additional proteins can be included in an expression vector. For example, such additional proteins include non-NeuroD1 proteins such as reporters, including, but not limited to, beta-galactosidase, green fluorescent protein, and antibiotic resistance reporters.

In particular embodiments, the recombinant expression vector encodes at least NeuroD1 of SEQ ID NO:2, a protein having at least 95% identity to SEQ ID NO:2, or a protein encoded by a nucleic acid sequence substantially identical to SEQ ID NO:1.

In particular embodiments, the recombinant expression vector encodes at least NeuroD1 of SEQ ID NO:4, a protein having at least 95% identity to SEQ ID NO:4, or a protein encoded by a nucleic acid sequence substantially identical to SEQ ID NO:2.

SEQ ID NO:9 is an example of a nucleic acid including CAG promoter operably linked to a nucleic acid encoding NeuroD1, and further including a nucleic acid sequence encoding EGFP and an enhancer, WPRE. An IRES separates the nucleic acid encoding NeuroD1 and the nucleic acid encoding EGFP. SEQ ID NO:9 is inserted into an expression vector for expression of NeuroD1 and the reporter gene EGFP. Optionally, the IRES and nucleic acid encoding EGFP are removed and the remaining CAG promoter and operably linked nucleic acid encoding NeuroD1 is inserted into an expression vector for expression of NeuroD1. The WPRE or another enhancer is optionally included.

Optionally, a reporter gene is included in a recombinant expression vector encoding NeuroD1 (and/or Dlx2). A reporter gene may be included to produce a peptide or protein that serves as a surrogate marker for expression of NeuroD1 (and/or Dlx2) from the recombinant expression vector. The term “reporter gene” as used herein refers to gene that is easily detectable when expressed, for example by chemiluminescence, fluorescence, colorimetric reactions, antibody binding, inducible markers, and/or ligand binding assays. Exemplary reporter genes include, but are not limited to, green fluorescent protein (GFP), enhanced green fluorescent protein (eGFP), yellow fluorescent protein (YFP), enhanced yellow fluorescent protein (eYFP), cyan fluorescent protein (CFP), enhanced cyan fluorescent protein (eCFP), blue fluorescent protein (BFP), enhanced blue fluorescent protein (eBFP), MmGFP (Zernicka-Goetz et al., Development, 124:1133-1137 (1997)), dsRed, luciferase, and beta-galactosidase (lacZ).

The process of introducing genetic material into a recipient host cell, such as for transient or stable expression of a desired protein encoded by the genetic material in the host cell is referred to as “transfection.” Transfection techniques are well-known in the art and include, but are not limited to, electroporation, particle accelerated transformation also known as “gene gun” technology, liposome-mediated transfection, calcium phosphate or calcium chloride co-precipitation-mediated transfection, DEAE-dextran-mediated transfection, microinjection, polyethylene glycol mediated transfection, heat shock mediated transfection, and virus-mediated transfection. As noted herein, virus-mediated transfection may be accomplished using a viral vector such as those derived from adenovirus, adeno-associated virus, and lentivirus.

Optionally, a host cell is transfected ex-vivo and then re-introduced into a host organism. For example, cells or tissues may be removed from a subject, transfected with an expression vector encoding NeuroD1 (and/or Dlx2) and then returned to the subject.

Introduction of a recombinant expression vector including a nucleic acid encoding NeuroD1, or a functional fragment thereof, and/or a nucleic acid encoding Dlx2, or a functional fragment thereof, into a host glial cell in vitro or in vivo for expression of exogenous NeuroD1 and/or Dlx2 in the host glial cell to convert the glial cell to a neuron is accomplished by any of various transfection methodologies.

Expression of exogenous NeuroD1 and/or Dlx2 in the host glial cell to convert the glial cell to a neuron is optionally achieved by introduction of mRNA encoding NeuroD1, or a functional fragment thereof, and/or mRNA encoding Dlx2, or a fragment thereof, to the host glial cell in vitro or in vivo.

Expression of exogenous NeuroD1 and/or Dlx2 in the host glial cell to convert the glial cell to a neuron is optionally achieved by introduction of NeuroD1 protein and/or Dlx2 protein to the host glial cell in vitro or in vivo. Details of these and other techniques are known in the art, for example, as described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; and Engelke, D. R., RNA Interference (RNAi): Nuts and Bolts of RNAi Technology, DNA Press LLC, Eagleville, P A, 2003.

An expression vector including a nucleic acid encoding NeuroD1 or a functional fragment thereof, and/or Dlx2 or a function fragment thereof, mRNA encoding NeuroD1 or a functional fragment thereof, and/or mRNA encoding Dlx2 or a functional fragment thereof, and/or NeuroD1 protein and/or Dlx2 protein, full-length or a functional fragment thereof, is optionally associated with a carrier for introduction into a host cell in vitro or in vivo.

In particular aspects, the carrier is a particulate carrier such as lipid particles including liposomes, micelles, unilamellar, or mulitlamellar vesicles; polymer particles such as hydrogel particles, polyglycolic acid particles, or polylactic acid particles; inorganic particles such as calcium phosphate particles such as those described elsewhere (e.g., U.S. Pat. No. 5,648,097); and inorganic/organic particulate carriers such as those described elsewhere (e.g., U.S. Pat. No. 6,630,486).

A particulate carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm to 10 microns. In particular aspects, a particulate carrier is formulated such that particles have an average particle size in the range of about 1 nm to 100 nm.

Further description of liposomes and methods relating to their preparation and use may be found in Liposomes: A Practical Approach (The Practical Approach Series, 264), V. P. Torchilin and V. Weissig (Eds.), Oxford University Press; 2nd ed., 2003. Further aspects of nanoparticles are described in S. M. Moghimi et al., FASEB J., 19:311-30 (2005).

Expression of NeuroD1 and/or Dlx2 using a recombinant expression vector is accomplished by introduction of the expression vector into a eukaryotic or prokaryotic host cell expression system such as an insect cell, mammalian cell, yeast cell, bacterial cell or any other single or multicellular organism recognized in the art. Host cells are optionally primary cells or immortalized derivative cells. Immortalized cells are those which can be maintained in vitro for at least 5 replication passages.

Host cells containing the recombinant expression vector are maintained under conditions wherein NeuroD1 and/or Dlx2 is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

In some cases, a recombinant expression vector including a nucleic acid encoding NeuroD1 and/or Dlx2 is introduced into glial cells of a subject. Expression of exogenous NeuroD1 and/or Dlx2 in the glial cells “converts” the glial cells into neurons.

In some cases, a recombinant expression vector including a nucleic acid encoding NeuroD1 and/or Dlx2 or a functional fragment thereof is introduced into astrocytes of a subject. Expression of exogenous NeuroD1 and/or exogenous Dlx2 in the glial cells “converts” the astrocytes into neurons.

In some cases, a recombinant expression vector including a nucleic acid encoding NeuroD1 and/or a nucleic acid encoding Dlx2, or a functional fragment thereof is introduced into reactive astrocytes of a subject. Expression of exogenous NeuroD1 and/or exogenous Dlx2, or a functional fragment thereof in the reactive astrocytes “converts” the reactive astrocytes into neurons.

In some cases, a recombinant expression vector including a nucleic acid encoding NeuroD1 and/or a nucleic acid encoding Dlx2, or a functional fragment thereof is introduced into NG2 cells of a subject. Expression of exogenous NeuroD1 and/or exogenous Dlx2, or a functional fragment thereof in the NG2 cells “converts” the NG2 cells into neurons.

Detection of expression of exogenous NeuroD1 and/or exogenous Dlx2 following introduction of a recombinant expression vector including a nucleic acid encoding the exogenous NeuroD1 and/or a nucleic acid encoding the exogenous Dlx2, or a functional fragment thereof is accomplished using any of various standard methodologies including, but not limited to, immunoassays to detect NeuroD1 and/or Dlx2, nucleic acid assays to detect NeuroD1 nucleic acids and/or Dlx2 nucleic acids, and detection of a reporter gene co-expressed with the exogenous NeuroD1 and/or exogenous Dlx2.

The terms “converts” and “converted” are used herein to describe the effect of expression of NeuroD1 or a functional fragment thereof and/or Dlx2 or a functional fragment thereof resulting in a change of a glial cell, astrocyte or reactive astrocyte phenotype to a neuronal phenotype. Similarly, the phrases “NeuroD1 converted neurons”, “Dlx2 converted neurons”, “NeuroD1 and Dlx2 converted neurons” and “converted neurons” are used herein to designate a cell including exogenous NeuroD1 protein or a functional fragment thereof which has consequent neuronal phenotype.

The term “phenotype” refers to well-known detectable characteristics of the cells referred to herein. The neuronal phenotype can be, but is not limited to, one or more of: neuronal morphology, expression of one or more neuronal markers, electrophysiological characteristics of neurons, synapse formation and release of neurotransmitter. For example, neuronal phenotype encompasses but is not limited to: characteristic morphological aspects of a neuron such as presence of dendrites, an axon and dendritic spines; characteristic neuronal protein expression and distribution, such as presence of synaptic proteins in synaptic puncta, presence of MAP2 in dendrites; and characteristic electrophysiological signs such as spontaneous and evoked synaptic events.

In a further example, glial phenotype such as astrocyte phenotype and reactive astrocyte phenotypes encompasses but is not limited to: characteristic morphological aspects of astrocytes and reactive astrocytes such as a generally “star-shaped” morphology; and characteristic astrocyte and reactive astrocyte protein expression, such as presence of glial fibrillary acidic protein (GFAP).

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

The term “NeuroD1 nucleic acid” refers to an isolated NeuroD1 nucleic acid molecule and encompasses isolated NeuroD1 nucleic acids having a sequence that has at least 70%, 75%, 80%, 85%, 90%, 91%, 92%, 93%, 94%, 95%, 96%, 97%, 98%, or 99% identity to the DNA sequence set forth in SEQ ID NO:1 or SEQ ID NO:3, or the complement thereof, or a fragment thereof, or an isolated DNA molecule having a sequence that hybridizes under high stringency hybridization conditions to the nucleic acid set forth as SEQ ID NO:1 or SEQ ID NO:3, a complement thereof or a fragment thereof.

The nucleic acid of SEQ ID NO:3 is an example of an isolated DNA molecule having a sequence that hybridizes under high stringency hybridization conditions to the nucleic acid set forth in SEQ ID NO:1. A fragment of a NeuroD1 nucleic acid is any fragment of a NeuroD1 nucleic acid that is operable in an aspect described herein including a NeuroD1 nucleic acid.

A nucleic acid probe or primer able to hybridize to a target NeuroD1 mRNA or cDNA can be used for detecting and/or quantifying mRNA or cDNA encoding NeuroD1 protein. A nucleic acid probe can be an oligonucleotide of at least 10, 15, 30, 50 or 100 nucleotides in length and sufficient to specifically hybridize under stringent conditions to NeuroD1 mRNA or cDNA or complementary sequence thereof. A nucleic acid primer can be an oligonucleotide of at least 10, 15 or 20 nucleotides in length and sufficient to specifically hybridize under stringent conditions to the mRNA or cDNA, or complementary sequence thereof.

The term “Dlx2 nucleic acid” refers to an isolated Dlx2 nucleic acid molecule and encompasses isolated Dlx2 nucleic acids having a sequence that has at least 70%, 75%, 80%, 85%, 90%, 91%, 92%, 93%, 94%, 95%, 96%, 97%, 98%, or 99% identity to the DNA sequence set forth in SEQ ID NO:10 or SEQ ID NO:12, or the complement thereof, or a fragment thereof, or an isolated DNA molecule having a sequence that hybridizes under high stringency hybridization conditions to the nucleic acid set forth as SEQ ID NO:10 or SEQ ID NO:12, a complement thereof or a fragment thereof.

The nucleic acid of SEQ ID NO:12 is an example of an isolated DNA molecule having a sequence that hybridizes under high stringency hybridization conditions to the nucleic acid set forth in SEQ ID NO:10. A fragment of a Dlx2 nucleic acid is any fragment of a Dlx2 nucleic acid that is operable in an aspect described herein including a Dlx2 nucleic acid.

A nucleic acid probe or primer able to hybridize to a target Dlx2 mRNA or cDNA can be used for detecting and/or quantifying mRNA or cDNA encoding Dlx2 protein. A nucleic acid probe can be an oligonucleotide of at least 10, 15, 30, 50 or 100 nucleotides in length and sufficient to specifically hybridize under stringent conditions to NeuroD1 mRNA or cDNA or complementary sequence thereof. A nucleic acid primer can be an oligonucleotide of at least 10, 15 or 20 nucleotides in length and sufficient to specifically hybridize under stringent conditions to the mRNA or cDNA, or complementary sequence thereof.

The terms “complement” and “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution.

Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/mL denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone. Under highly stringent conditions, SEQ ID NO:1 and SEQ ID NO:3 will hybridize to the complement of substantially identical targets and not to unrelated sequences.

Methods of treating a neurological condition in a subject in need thereof are provided according to some aspects described herein which include delivering a therapeutically effective amount of NeuroD1 and/or Dlx2 to glial cells of the central nervous system or peripheral nervous system of the subject, the therapeutically effective amount of NeuroD1 and/or Dlx2 in the glial cells results in a greater number of neurons in the subject compared to an untreated subject having the same neurological condition, whereby the neurological condition is treated.

The conversion of reactive glial cells into neurons also reduces neuroinflammation and neuroinhibitory factors associated with reactive glial cells, thereby making the glial scar tissue more permissive to neuronal growth so that neurological condition is alleviated.

The term “neurological condition” or “neurological disorder” as used herein refers to any condition of the central nervous system of a subject which is alleviated, ameliorated or prevented by additional neurons. Injuries or diseases which result in loss or inhibition of neurons and/or loss or inhibition of neuronal function are neurological conditions for treatment by methods described herein.

Injuries or diseases which result in loss or inhibition of glutamatergic neurons and/or loss or inhibition of glutaminergic neuronal functions are neurological conditions that can be treated as described herein. Loss or inhibition of other types of neurons, such as GABAergic, cholinergic, dopaminergic, norepinephrinergic, or serotonergic neurons can be treated with the similar method.

The term “therapeutically effective amount” as used herein is intended to mean an amount of an inventive composition which is effective to alleviate, ameliorate or prevent a symptom or sign of a neurological condition to be treated. In particular embodiments, a therapeutically effective amount is an amount which has a beneficial effect in a subject having signs and/or symptoms of a neurological condition.

The terms “treat,” “treatment,” “treating,” “NeuroD1 treatment,” “Dlx2 treatment” and “NeuroD1 and Dlx2 treatment” or grammatical equivalents as used herein refer to alleviating, inhibiting or ameliorating a neurological condition, symptoms or signs of a neurological condition, and preventing symptoms or signs of a neurological condition, and include, but are not limited to therapeutic and/or prophylactic treatments.

Signs and symptoms of neurological conditions are well-known in the art along with methods of detection and assessment of such signs and symptoms.

In some cases, combinations of therapies for a neurological condition of a subject can be administered.

According to particular aspects an additional pharmaceutical agent or therapeutic treatment administered to a subject to treats the effects of disruption of normal blood flow in the CNS in an individual subject in need thereof include treatments such as, but not limited to, removing a blood clot, promoting blood flow, administration of one or more anti-inflammation agents, administration of one or more anti-oxidant agents, and administration of one or more agents effective to reduce excitotoxicity

The term “subject” refers to humans and also to non-human mammals such as, but not limited to, non-human primates, cats, dogs, sheep, goats, horses, cows, pigs and rodents, such as but not limited to, mice and rats; as well as to non-mammalian animals such as, but not limited to, birds, poultry, reptiles, amphibians.

Embodiments of inventive compositions and methods are illustrated in the following examples. These examples are provided for illustrative purposes and are not considered limitations on the scope of inventive compositions and methods.

### EXAMPLES

### Example 1—Histology of Intracerebral Hemorrhage

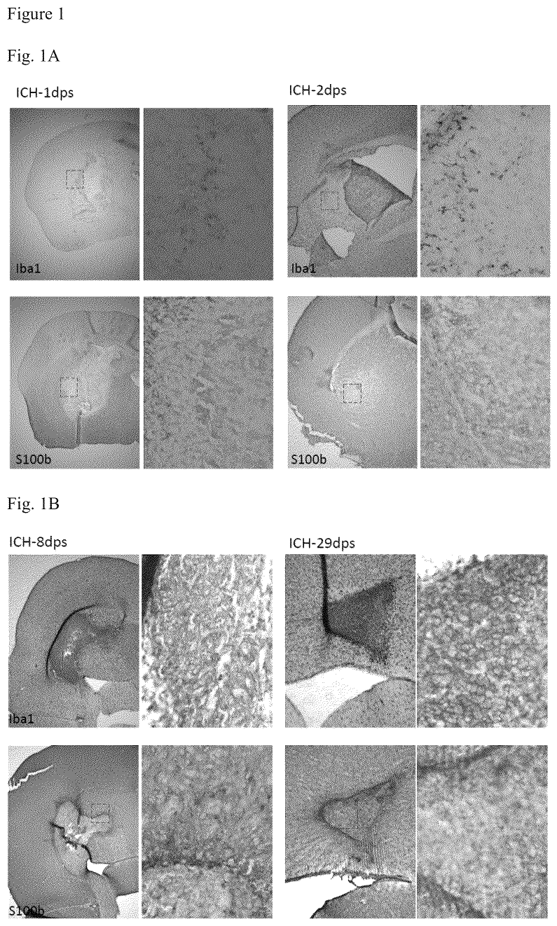

0.2 μL collagenase was injected to mouse striatum. After 1 day, 2 days, 8 days, and 29 days, data were collected, and DAB and iron staining were conducted. FIG. 1A and FIG. 1B showed the Iba1 and S100b DAB staining with iron staining from 1 day to 29 days post induction of ICH.

These results showed the morphological changes of astrocytes and microglia after ICH as well as the process of accumulation of ferric iron. These results provided a reference to choose time points to intervene to treat ICH.

### Example 2—In Vivo Conversion of Reactive Astrocytes to Neurons in a Mouse Model of Intracerebral Hemorrhage (Short Term)

A set of experiments was performed to assess the in vivo conversion of reactive astrocytes into neurons following treatment with AAV5 viruses encoding NeuroD1 and Dlx2. ICH induction at day 0 was performed by injecting 0.2 μL of collagenase into striatum. Mice were injected with 1 μL of AAV5-GFA104-cre: 3×10¹¹, 1 μL of AAV5-CAG-flex-GFP: 3.4×10¹¹, 1 μL of AAV5-CAG-flex-ND1-GFP: 4.55×10¹¹, or 1 μL of AAV5-CAG-flex-Dlx2-GFP: 2.36×10¹² at 2 days, 4 days, and 7 days post ICH induction. On day 21, data regarding astrocyte conversion were collected.

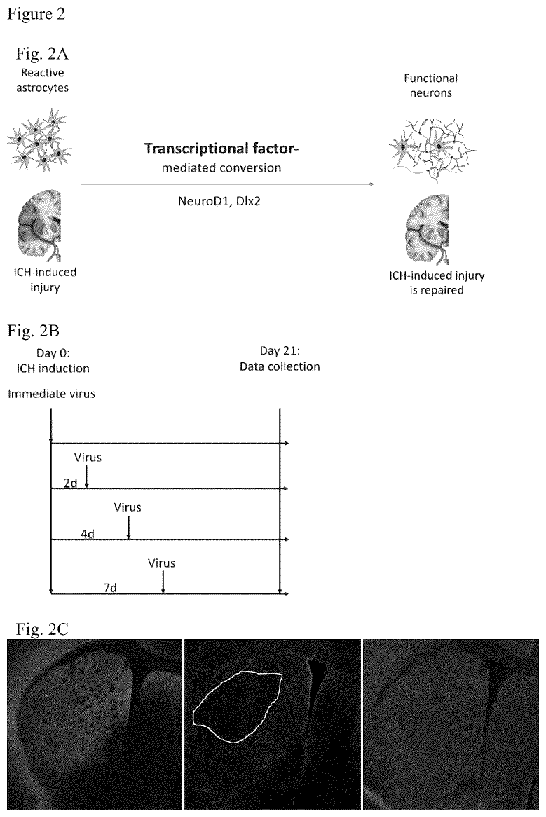

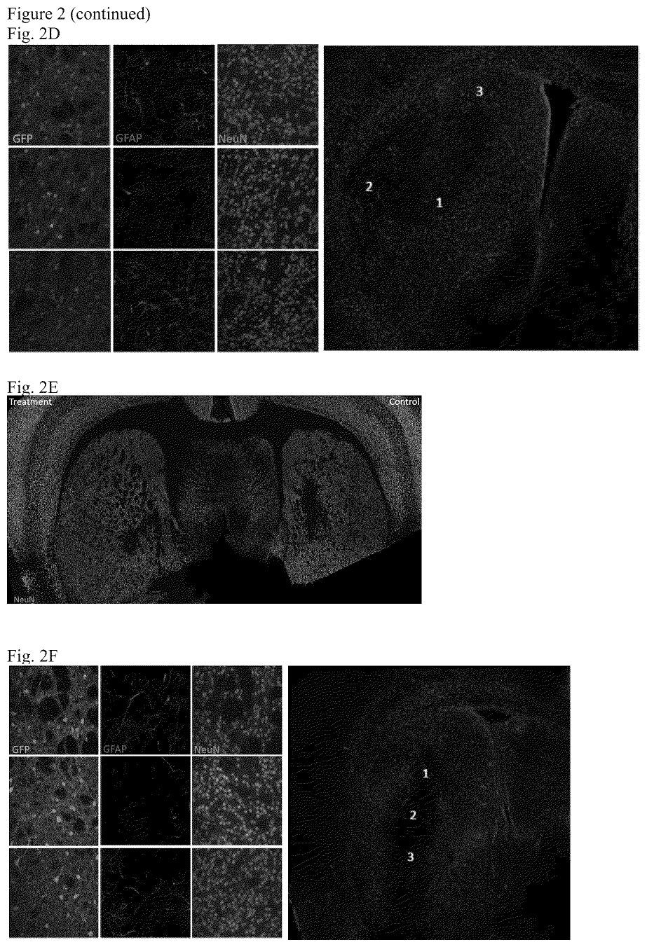

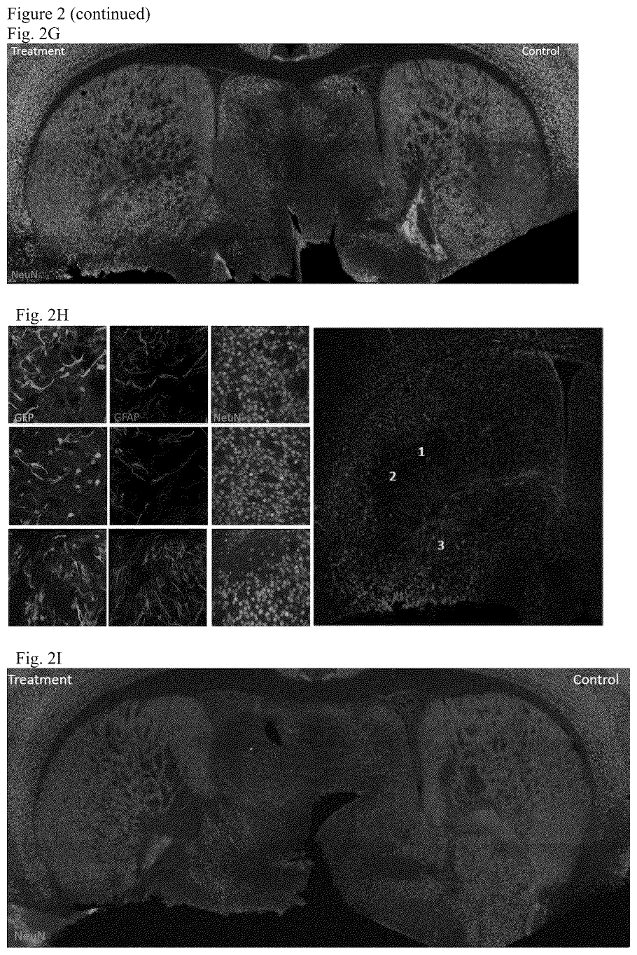

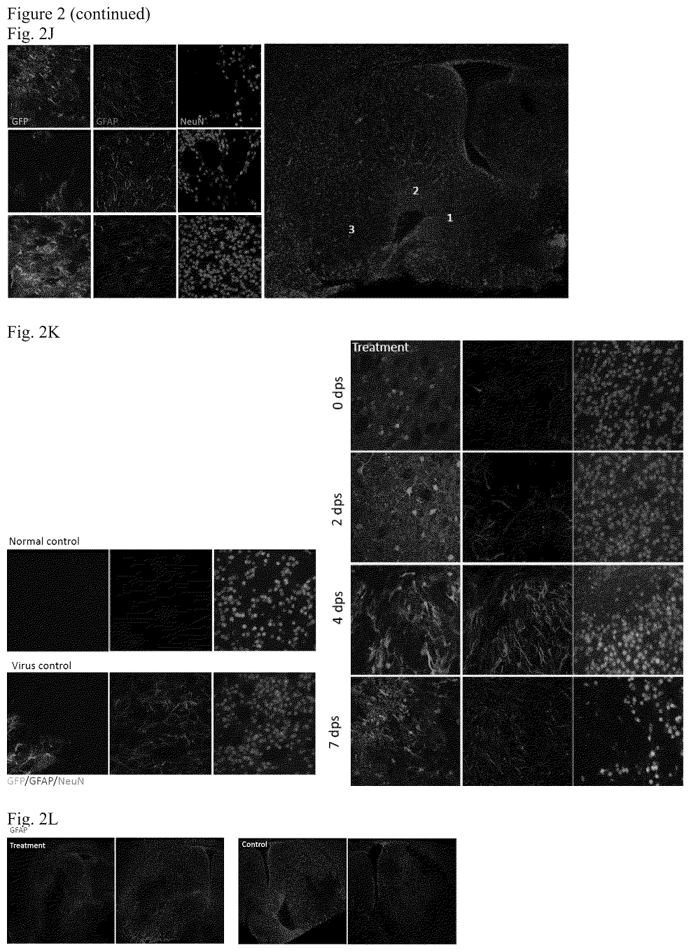

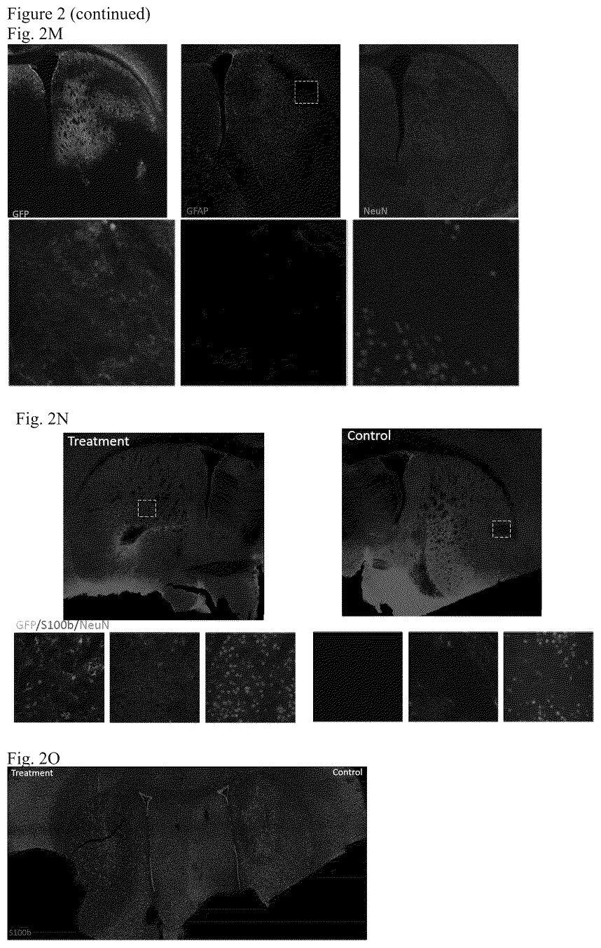

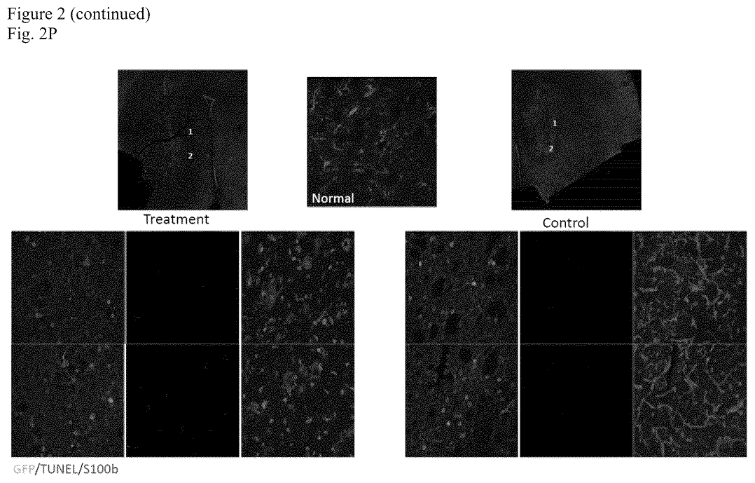

FIG. 2A and FIG. 2B showed the schematics of the experiments about in vivo conversion in short term. Different virus injection times (immediately, 2 dps, 4 dps, and 7 dps) were conducted to find the optimal time window to repair ICH. FIG. 2C, FIG. 2D, FIG. 2E, FIG. 2F, FIG. 2G, FIG. 2H, FIG. 2I, FIG. 2J, FIG. 2K, FIG. 2L, FIG. 2M, FIG. 2N, FIG. 2O, and FIG. 2P revealed the immunostaining of GFP, GFAP, and NeuN, accordingly. The results consistently showed the decrease of conversion, decrease of neuronal density, and increase of reactive astrocytes around the injury core along with the delay of virus injection time point.

These results demonstrate that earlier virus injection has a better treatment effect. If virus is injected immediately or within 2 days after stroke, a higher conversion rate can be achieved, and astrocytes would be less reactive.

### Example 3—In Vivo Conversion of Reactive Astrocytes to Neurons in a Mouse Model of Intracerebral Hemorrhage (Long Term)

A set of experiments was performed to assess the in vivo conversion of reactive astrocytes into neurons following treatment with AAV5 viruses encoding NeuroD1 and Dlx2. ICH induction at day 0 was performed by injecting 0.35 μL of collagenase into striatum. Mice were injected with 1 μL of AAV5-GFA104-cre: 3×10¹¹, 1 μL of AAV5-CAG-flex-GFP: 3.4×10¹¹, 1 μL of AAV5-CAG-flex-ND1-GFP: 4.55×10¹¹, or 1 μL of AAV5-CAG-flex-Dlx2-GFP: 2.36×10¹² at 2 days and 7 days post ICH induction. Two months post induction, mice were harvested, and data were collected.

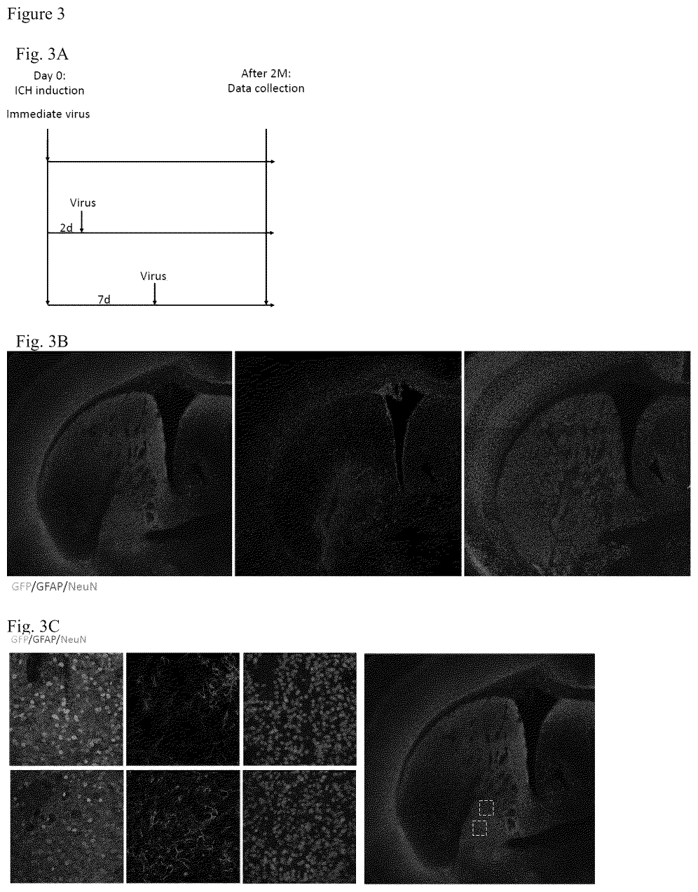

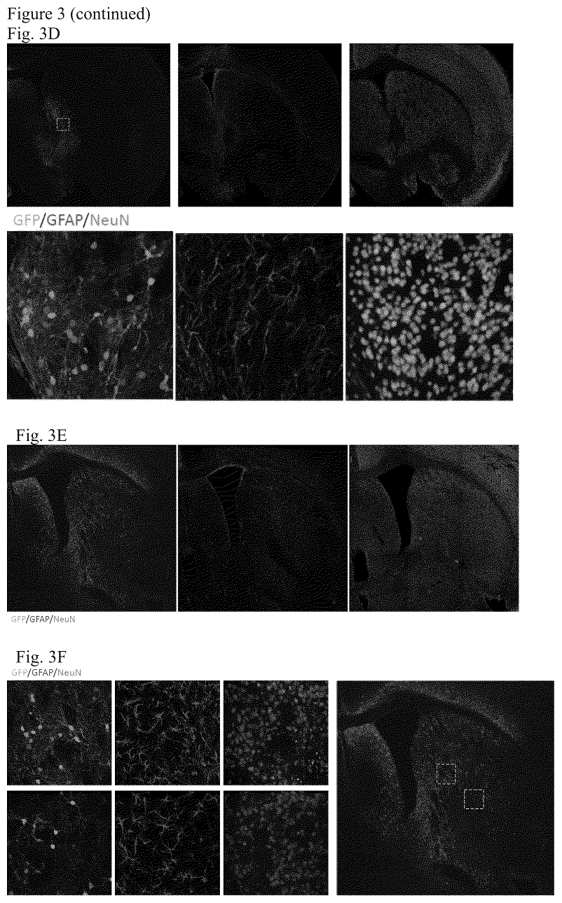

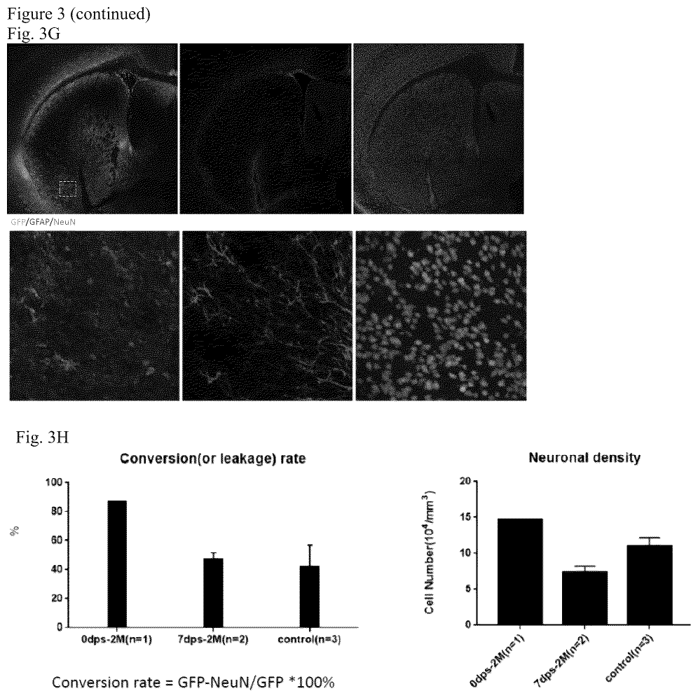

FIG. 3A shows the experimental design of the long-term repair effect of ND1 and Dlx2 on ICH. FIG. 3B, FIG. 3C, FIG. 3D, FIG. 3E, FIG. 3F, and FIG. 3G present the immunostaining of GFP, GFAP, and NeuN. FIG. 3B and FIG. 3C showed almost all the GFP-positive cells had neuronal morphologies and expressed NeuN two months after virus infection when the virus was injected immediately after ICH. FIG. 3D showed the 2 months of virus infection when the virus was injected 2 days after ICH. The infection was not wide, which might be caused by the virus injection point being too close to the ventricle. FIG. 3E and FIG. 3F showed the immunostaining after 2 months of virus infection after it was injected 7 days after ICH. The conversion rate was lower than immediate virus injection after ICH. FIG. 3H showed the comparison of conversion rate and neuronal density for different virus injection time points (2 dps was excluded for low infection). It showed immediate virus injection might be an ideal time point for treating ICH.

These results demonstrate that earlier virus injection after ICH might have a better repair outcome: higher conversion rate and higher neuronal density.

### Example 4—Evaluation on Viral Vector in In Vivo Conversion after ICH: AAV9-1.6 kb-GFAP-Cre-Flex System

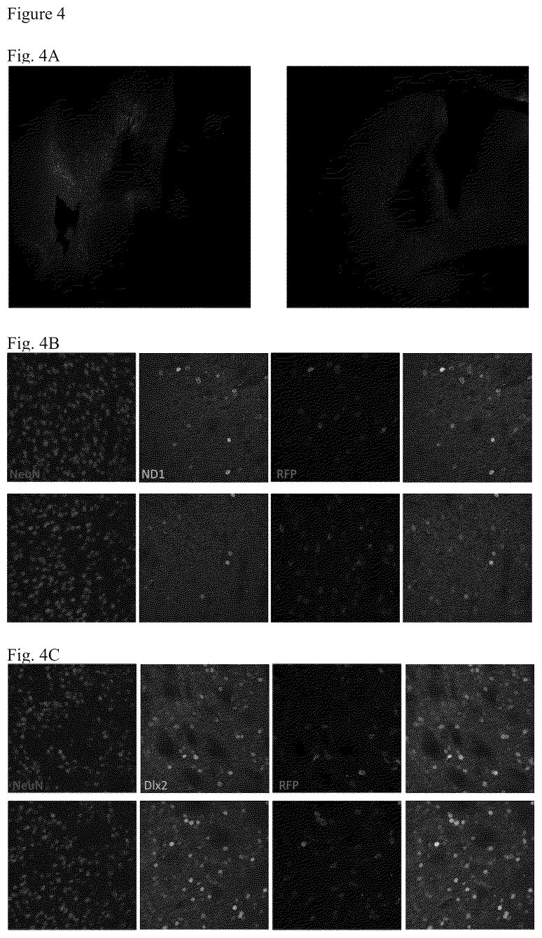

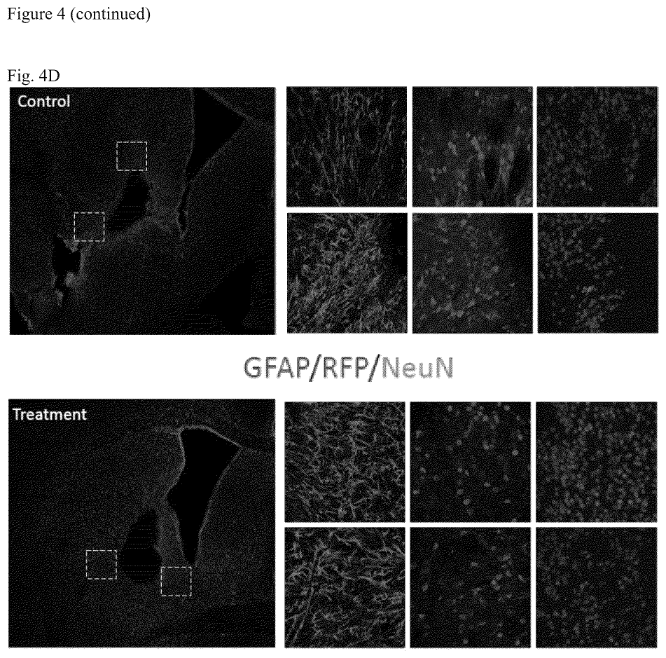

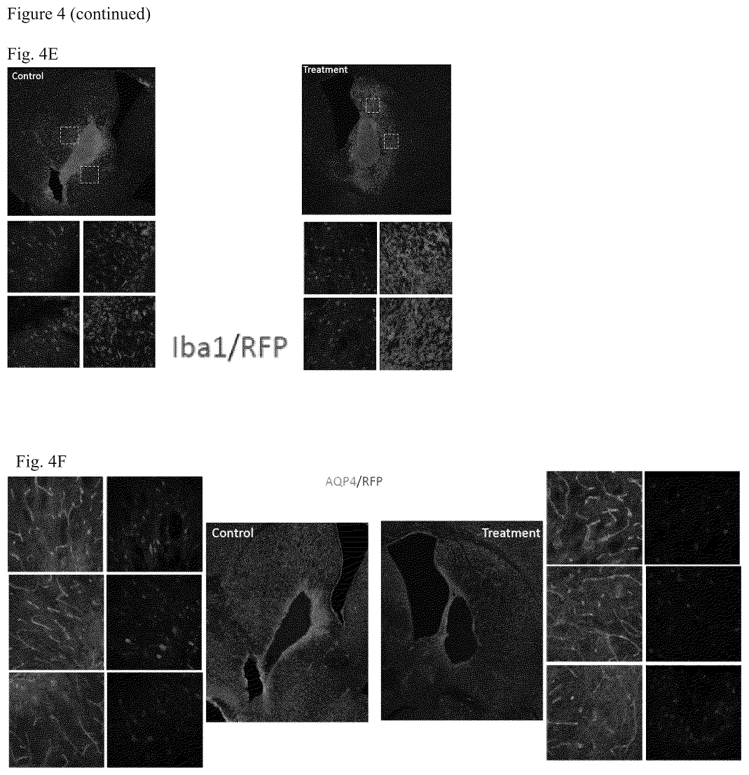

To achieve a higher infection and higher expression of ND1 and Dlx2, the following viral system was developed: AAV9-1.6 kb-GFAP-cre with flex-ND1-mCherry and flex-Dlx2-mCherry. The results in FIG. 4A, FIG. 4B, FIG. 4C, FIG. 4D, FIG. 4E, and FIG. 4F suggest that even though AAV9 can achieve a higher expression of ND1 and Dlx2, it has more leakage than AAV5. However, the treatment still showed less dense glia scar reflected by GFAP, and slightly better morphologies of blood vessel showed in AQP4. The Iba1 signal was stronger in treatment than control, while the role of microglia in conversion was unclear.

These results demonstrate that regardless of leakage, AAV9-1.6 kb-GFAP-cre-flex system can be an effective alternative for in vivo astrocyte to neuron conversion after ICH.

### Example 5—Evaluation on Viral Vector in In Vivo Conversion after ICH: AAV5-1.6 kb-GFAP-Cre-Flex System and the Effect of Injury on Conversion Rate

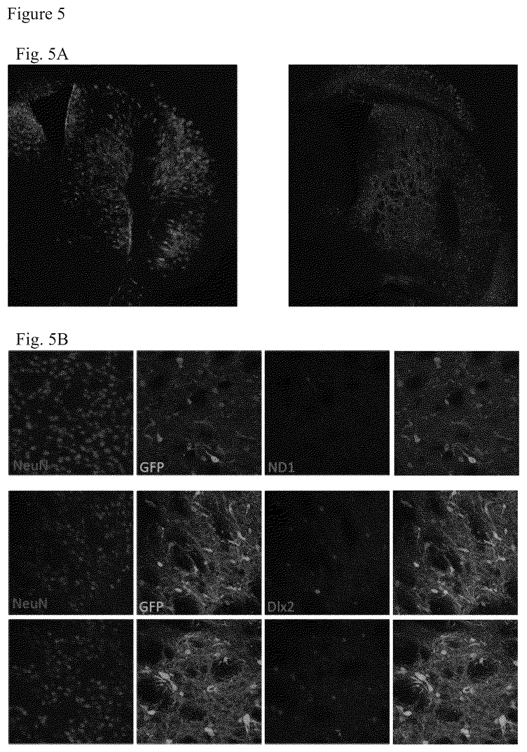

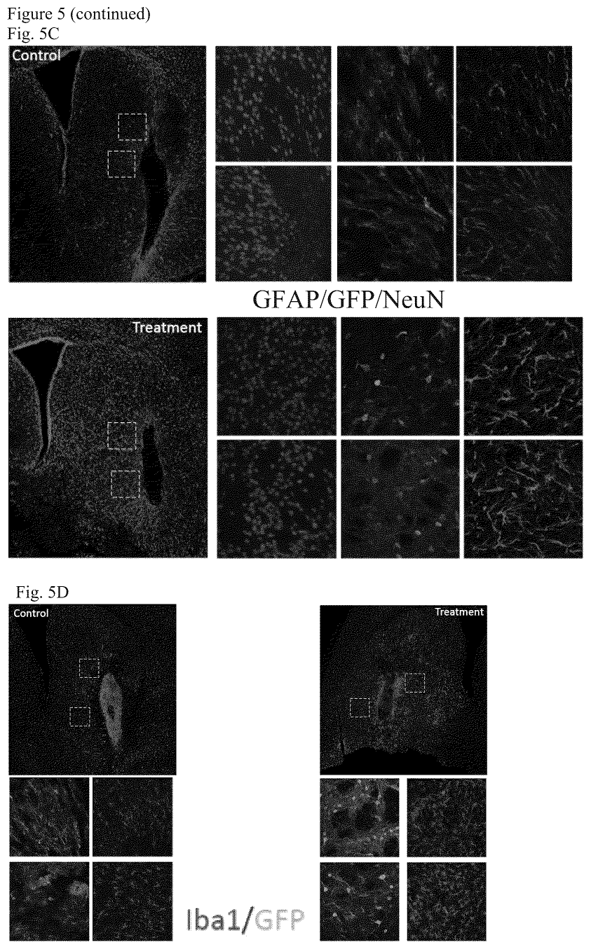

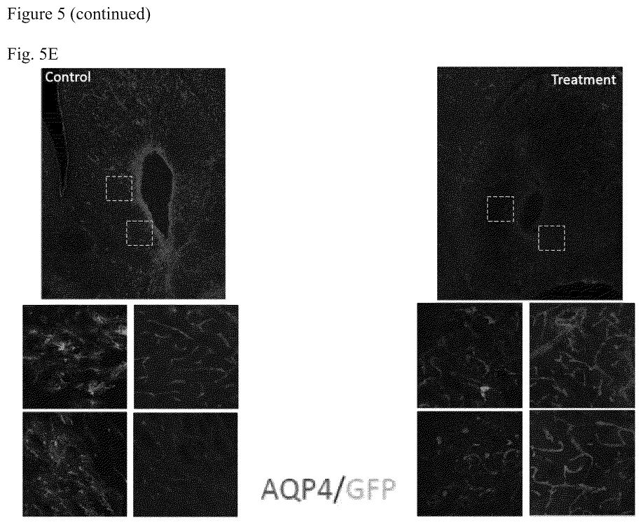

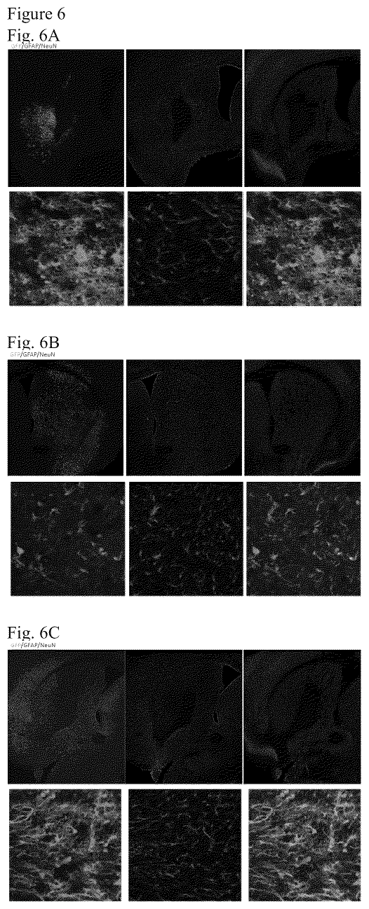

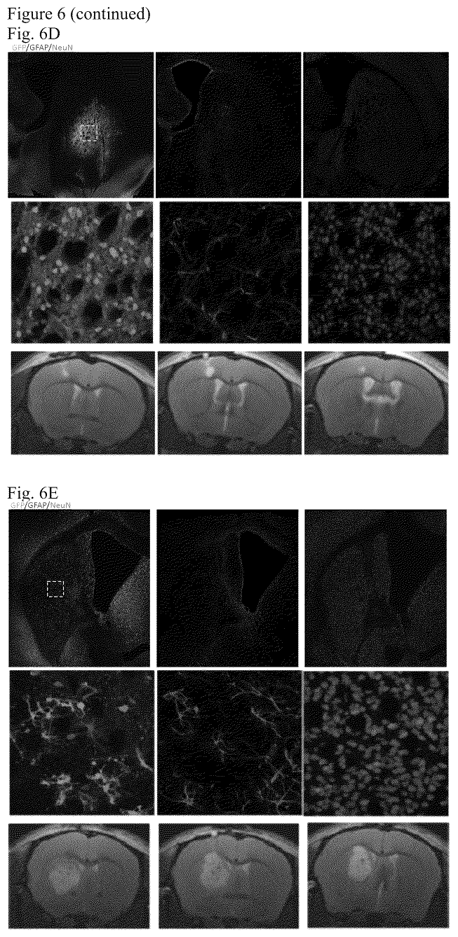

FIG. 5A, FIG. 5B, FIG. 5C, FIG. 5D, and FIG. 5E showed the infection by AAV5 system. There were few neurons that were GFP-positive, indicating this system is relatively clean. Besides, the recovery effect was observed in different aspects: the downregulation of GFAP signal around injury core, the increase of neuronal density, and more AQP4 signal around blood vessels suggesting recovery of blood-brain-barrier. This indicated that AAV5 system is an effective system for in vivo astrocyte to neuron conversion and treatment for ICH. FIG. 6A, FIG. 6B, FIG. 6C, FIG. 6D, and FIG. 6E showed the effect of injury on conversion rate. The more severe the injury was, the lower the conversion rate was.

### Example 6—Reasoning of the Ideal Time Point for Treatment Application for In Vivo Conversion after ICH

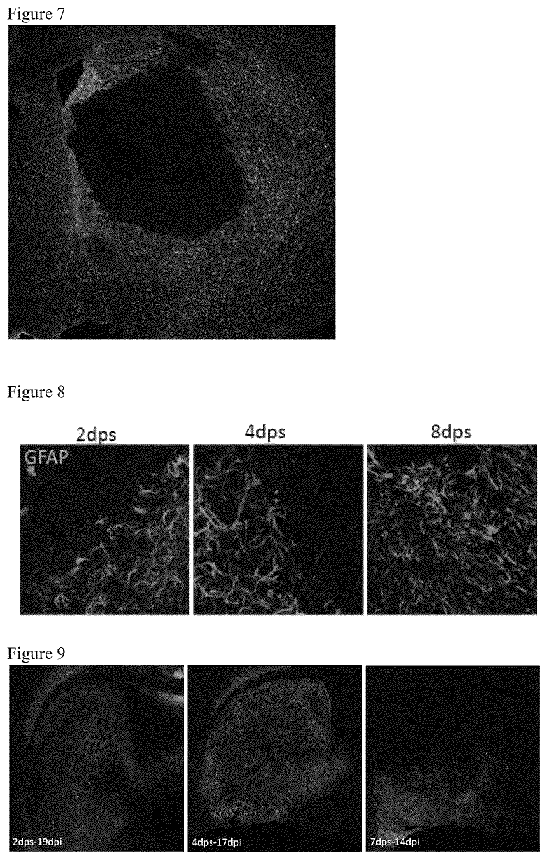

FIG. 7 showed that the virus infection for 4 days at 2 days after collagenase injection. The hematoma was visible, and there was no virus signal within the hematoma. There was significant viral infection at the surrounding area of the hematoma. It was possible that the existence of the hematoma hindered the virus infection and repair after ICH. To resolve this issue, one or more small molecules can be administered to inhibit the growth of the hematoma and/or the virus(es) can be administered one or more additional times after the hematoma is absorbed to get improved expression of ND1 and Dlx2.

FIG. 8 revealed the it is beneficial to take action soon when ICH occurs. Astrocytes started to proliferate after ICH and reach the peak around 5 dps. FIG. 8 also revealed that the dense glia scar formed at 8 dps. Glia scar isolated the injury core and made the injury irreversible. Thus, to avoid the formation of glia scar, treatment can be apply as soon as possible (e.g., less than 5 dps, less than 4 dps, less than 3 dps, less than 2 dps, less than 1 dps, within 12 hours of stroke, within 8 hours of stroke, or within 6 hours of stroke).

### Example 7—Miscellaneous Materials

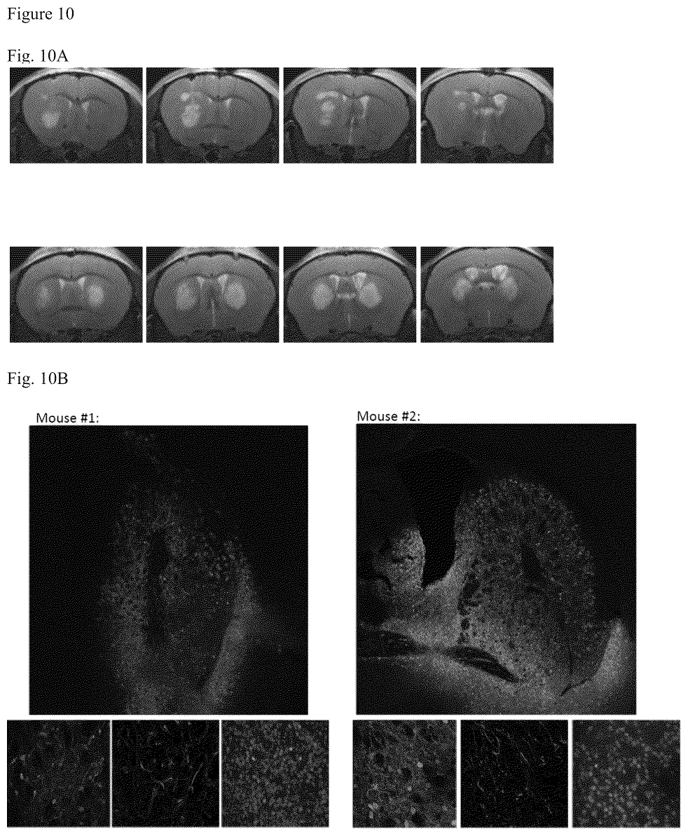

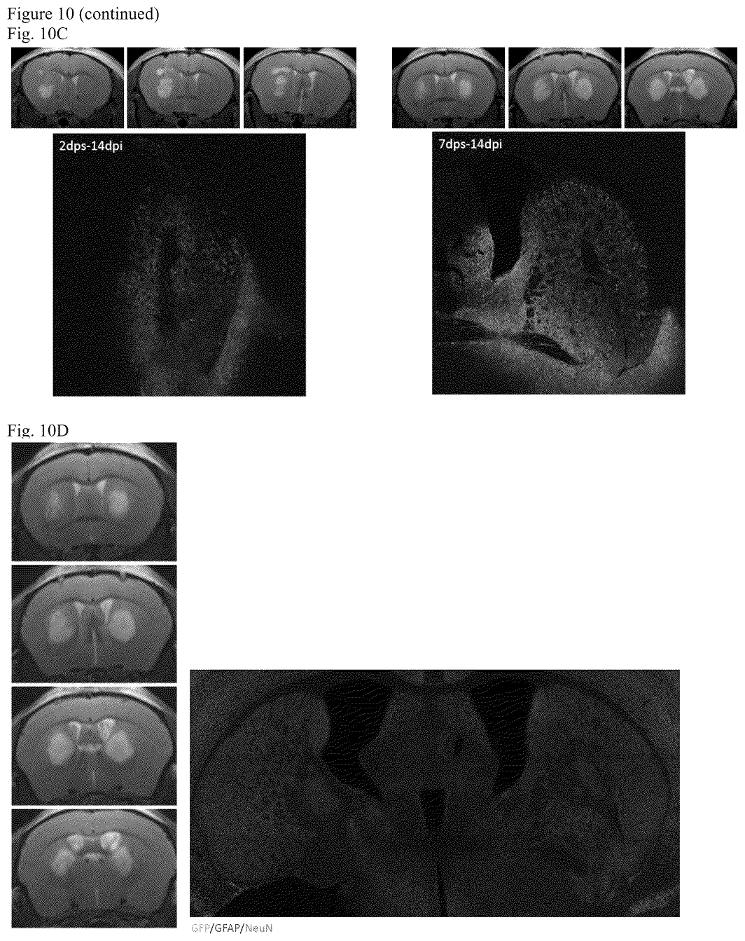

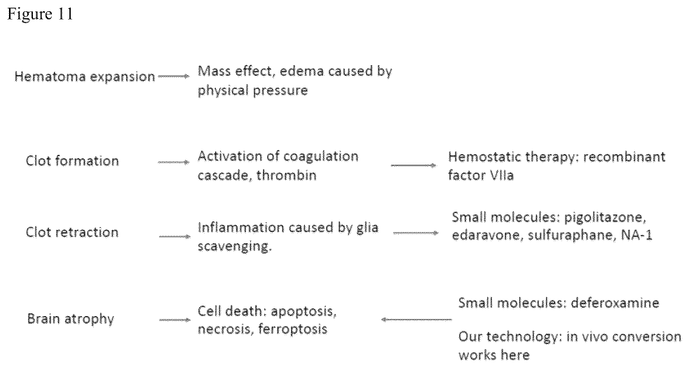

FIG. 9 showed that early virus injection can lead to smaller size of injury core and higher conversion rate. FIG. 10 showed the rare situation that virus injection at 7 dps might be better than 2 dps. However, the initial conditions were measured at different time points after ICH. FIG. 11 showed a simple diagram of the process of ICH and the corresponding treatments for each step. The technology can be used for long-term recovery after ICH.

### Example 8—Additional Embodiments

Embodiment 1. A method for (1) generating new glutamatergic neurons, (2) increasing survival of GABAergic neurons, (3) generating new non-reactive astrocytes, or (4) reducing the number of reactive astrocytes, in a mammal having had a hemorrhagic stroke and in need of (1), (2), (3), or (4), wherein said method comprises administering a composition comprising exogenous nucleic acid encoding a Neurogenic Differentiation 1 (NeuroD1) polypeptide or a biologically active fragment thereof and exogenous nucleic acid encoding a Distal-less homeobox 2 (Dlx2) polypeptide or a biologically active fragment thereof to said mammal.

Embodiment 2. The method of embodiment 1, wherein said mammal is a human.

Embodiment 3. The method of embodiment 1, wherein the hemorrhagic stroke is due to a condition selected from the group consisting of: ischemic stroke; physical injury; tumor; inflammation; infection; global ischemia as caused by cardiac arrest or severe hypotension (shock); hypoxic-ischemic encephalopathy as caused by hypoxia, hypoglycemia, or anemia; meningitis; and dehydration; or a combination of any two or more thereof.

Embodiment 4. The method of embodiment 1, wherein said administering step comprises delivering an expression vector comprising a nucleic acid encoding a NeuroD1 polypeptide or a biologically active fragment thereof and an expression vector comprising a nucleic acid encoding a Dlx2 polypeptide or a biologically active fragment thereof to the location of the hemorrhagic stroke in the brain.

Embodiment 5. The method of embodiment 1 or 2, wherein said administering step comprises delivering a recombinant viral expression vector comprising a nucleic acid encoding a NeuroD1 polypeptide or a biologically active fragment thereof and a recombinant viral expression vector comprising a nucleic acid encoding a Dlx2 polypeptide or a biologically active fragment thereof to the location of the hemorrhagic stroke in the brain.

Embodiment 6. The method of any of embodiments 1-3, wherein said administering step comprises delivering a recombinant adeno-associated virus expression vector comprising a nucleic acid encoding a NeuroD1 polypeptide or a biologically active fragment thereof and a recombinant adeno-associated virus expression vector comprising a nucleic acid encoding a Dlx2 polypeptide or a biologically active fragment thereof to the location of the hemorrhagic stroke in the brain.

Embodiment 7. The method of any of embodiments 1-6, wherein said administering step comprises a stereotactic intracranial injection to the location of the hemorrhagic stroke in the brain.

Embodiment 8. The method of any one of embodiments 1-7, wherein said administering step further comprises administering the exogenous nucleic acid encoding a NeuroD1 polypeptide or a biologically active fragment thereof and exogenous nucleic acid encoding a Dlx2 polypeptide or a biologically active fragment thereof on one expression vector, one recombinant viral expression vector, or one recombinant adeno-associated virus expression vector.

Embodiment 9. The method of embodiment 1, wherein the composition comprises about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof and a nucleic acid encoding a Dlx2 polypeptide or a biologically active fragment thereof.

Embodiment 10. The method of embodiment 9, wherein the composition is injected in the brain of said mammal at a controlled flow rate of about 0.1 μL/minute to about 5 μL/minute.

Embodiment 11. A method for (1) generating new GABAergic and glutamatergic neurons, (2) increasing survival of GABAergic and glutamatergic neurons, (3) generating new non-reactive astrocytes, or (4) reducing the number of reactive astrocytes, in a mammal having had a hemorrhagic stroke and in need of (1), (2), (3), or (4), wherein said method comprises administering a composition comprising exogenous nucleic acid encoding a Neurogenic Differentiation 1 (NeuroD1) polypeptide or a biologically active fragment thereof and exogenous nucleic acid encoding a Distal-less homeobox 2 (Dlx2) polypeptide or a biologically active fragment thereof to said mammal within 3 days of said hemorrhagic stroke.

Embodiment 12. The method of embodiment 11, wherein said mammal is a human.

Embodiment 13. The method of embodiment 11, wherein the hemorrhagic stroke is due to a condition selected from the group consisting of: bleeding in the brain; aneurysm; intracranial hematoma; subarachnoid hemorrhage; brain trauma; high blood pressure; weak blood vessels; malformation of blood vessels; ischemic stroke; physical injury; tumor; inflammation; infection; global ischemia as caused by cardiac arrest or severe hypotension (shock); hypoxic-ischemic encephalopathy as caused by hypoxia, hypoglycemia, or anemia; meningitis; and dehydration; or a combination of any two or more thereof.

Embodiment 14. The method of embodiment 11, wherein said administering step comprises delivering an expression vector comprising a nucleic acid encoding a NeuroD1 polypeptide or a biologically active fragment thereof and an expression vector comprising a nucleic acid encoding a Dlx2 polypeptide or a biologically active fragment thereof to the location of the hemorrhagic stroke in the brain.

Embodiment 15. The method of embodiment 11 or 12, wherein said administering step comprises delivering a recombinant viral expression vector comprising a nucleic acid encoding a NeuroD1 polypeptide or a biologically active fragment thereof and a recombinant viral expression vector comprising a nucleic acid encoding a Dlx2 polypeptide or a biologically active fragment thereof to the location of the hemorrhagic stroke in the brain.

Embodiment 16. The method of any of embodiments 11-13, wherein said administering step comprises delivering a recombinant adeno-associated virus expression vector comprising a nucleic acid encoding a NeuroD1 polypeptide or a biologically active fragment thereof and a recombinant adeno-associated virus expression vector comprising a nucleic acid encoding a Dlx2 polypeptide or a biologically active fragment thereof to the location of the hemorrhagic stroke in the brain.

Embodiment 17. The method of any of embodiments 11-16, wherein said administering step comprises a stereotactic intracranial injection to the location of the hemorrhagic stroke in the brain.

Embodiment 18. The method of any one of embodiments 11-17, wherein said administering step further comprises administering the exogenous nucleic acid encoding a NeuroD1 polypeptide or a biologically active fragment thereof and exogenous nucleic acid encoding a Dlx2 polypeptide or a biologically active fragment thereof on one expression vector, one recombinant viral expression vector, or one recombinant adeno-associated virus expression vector.

Embodiment 19. The method of embodiment 11, wherein the composition comprises about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof and a nucleic acid encoding a Dlx2 polypeptide or a biologically active fragment thereof.

Embodiment 20. The method of embodiment 19, wherein the composition is injected in the brain of said mammal at a controlled flow rate of about 0.1 μL/minute to about 5 L/minute.

SEQUENCES

SEQ ID NO: 1
Human NeuroD1 nucleic acid sequence encoding human NeuroD1
protein - 1071 nucleotides, including stop codon
ATGACCAAATCGTACAGCGAGAGTGGGCTGATGGGCGAGCCTCAGCCCCAAGGTCCTCCAAGCT
GGACAGACGAGTGTCTCAGTTCTCAGGACGAGGAGCACGAGGCAGACAAGAAGGAGGACGACCT
CGAAGCCATGAACGCAGAGGAGGACTCACTGAGGAACGGGGGAGAGGAGGAGGACGAAGATGAG
GACCTGGAAGAGGAGGAAGAAGAGGAAGAGGAGGATGACGATCAAAAGCCCAAGAGACGCGGCC
CCAAAAAGAAGAAGATGACTAAGGCTCGCCTGGAGCGTTTTAAATTGAGACGCATGAAGGCTAA
CGCCCGGGAGCGGAACCGCATGCACGGACTGAACGCGGCGCTAGACAACCTGCGCAAGGTGGTG
CCTTGCTATTCTAAGACGCAGAAGCTGTCCAAAATCGAGACTCTGCGCTTGGCCAAGAACTACA
TCTGGGCTCTGTCGGAGATCCTGCGCTCAGGCAAAAGCCCAGACCTGGTCTCCTTCGTTCAGAC
GCTTTGCAAGGGCTTATCCCAACCCACCACCAACCTGGTTGCGGGCTGCCTGCAACTCAATCCT
CGGACTTTTCTGCCTGAGCAGAACCAGGACATGCCCCCCCACCTGCCGACGGCCAGCGCTTCCT
TCCCTGTACACCCCTACTCCTACCAGTCGCCTGGGCTGCCCAGTCCGCCTTACGGTACCATGGA
CAGCTCCCATGTCTTCCACGTTAAGCCTCCGCCGCACGCCTACAGCGCAGCGCTGGAGCCCTTC
TTTGAAAGCCCTCTGACTGATTGCACCAGCCCTTCCTTTGATGGACCCCTCAGCCCGCCGCTCA
GCATCAATGGCAACTTCTCTTTCAAACACGAACCGTCCGCCGAGTTTGAGAAAAATTATGCCTT
TACCATGCACTATCCTGCAGCGACACTGGCAGGGGCCCAAAGCCACGGATCAATCTTCTCAGGC
ACCGCTGCCCCTCGCTGCGAGATCCCCATAGACAATATTATGTCCTTCGATAGCCATTCACATC
ATGAGCGAGTCATGAGTGCCCAGCTCATGCCATATTTCATGATTAG
SEQ ID NO: 2
Human NeuroD1 amino acid sequence - 356 amino acids - encoded
by SEQ ID NO: 1
MTKSYSESGLMGEPQPQGPPSWTDECLSSQDEEHEADKKEDDLEAMNAEEDSLRNGGEEEDEDE
DLEEEEEEEEEDDDQKPKRRGPKKKKMTKARLERFKLRRMKANARERNRMHGLNAALDNLRKVV
PCYSKTQKLSKIETLRLAKNYIWALSEILRSGKSPDLVSFVQTLCKGLSQPTTNLVAGCLQLNP
RTFLPEQNQDMPPHLPTASASFPVHPYSYQSPGLPSPPYGTMDSSHVFHVKPPPHAYSAALEPF
FESPLTDCTSPSFDGPLSPPLSINGNFSFKHEPSAEFEKNYAFTMHYPAATLAGAQSHGSIFSG
TAAPRCEIPIDNIMSFDSHSHHERVMSAQLNAIFHD
SEQ ID NO: 3
Mouse NeuroD1 nucleic acid sequence encoding mouse NeuroD1
protein - 1074 nucleotides, including stop codon
ATGACCAAATCATACAGCGAGAGCGGGCTGATGGGCGAGCCTCAGCCCCAAGGTCCCCCAAGCT
GGACAGATGAGTGTCTCAGTTCTCAGGACGAGGAACACGAGGCAGACAAGAAAGAGGACGAGCT
TGAAGCCATGAATGCAGAGGAGGACTCTCTGAGAAACGGGGGAGAGGAGGAGGAGGAAGATGAG
GATCTAGAGGAAGAGGAGGAAGAAGAAGAGGAGGAGGAGGATCAAAAGCCCAAGAGACGGGGTC
CCAAAAAGAAAAAGATGACCAAGGCGCGCCTAGAACGTTTTAAATTAAGGCGCATGAAGGCCAA
CGCCCGCGAGCGGAACCGCATGCACGGGCTGAACGCGGCGCTGGACAACCTGCGCAAGGTGGTA
CCTTGCTACTCCAAGACCCAGAAACTGTCTAAAATAGAGACACTGCGCTTGGCCAAGAACTACA
TCTGGGCTCTGTCAGAGATCCTGCGCTCAGGCAAAAGCCCTGATCTGGTCTCCTTCGTACAGAC
GCTCTGCAAAGGTTTGTCCCAGCCCACTACCAATTTGGTCGCCGGCTGCCTGCAGCTCAACCCT
CGGACTTTCTTGCCTGAGCAGAACCCGGACATGCCCCCGCATCTGCCAACCGCCAGCGCTTCCT
TCCCGGTGCATCCCTACTCCTACCAGTCCCCTGGACTGCCCAGCCCGCCCTACGGCACCATGGA
CAGCTCCCACGTCTTCCACGTCAAGCCGCCGCCACACGCCTACAGCGCAGCTCTGGAGCCCTTC
TTTGAAAGCCCCCTAACTGACTGCACCAGCCCTTCCTTTGACGGACCCCTCAGCCCGCCGCTCA
GCATCAATGGCAACTTCTCTTTCAAACACGAACCATCCGCCGAGTTTGAAAAAAATTATGCCTT
TACCATGCACTACCCTGCAGCGACGCTGGCAGGGCCCCAAAGCCACGGATCAATCTTCTCTTCC
GGTGCCGCTGCCCCTCGCTGCGAGATCCCCATAGACAACATTATGTCTTTCGATAGCCATTCGC
ATCATGAGCGAGTCATGAGTGCCCAGCTTAATGCCATCTTTCACGATTAG
SEQ ID NO: 4
Mouse NeuroD1 amino acid sequence - 357 amino acids - encoded
by SEQ ID NO: 3
MTKSYSESGLMGEPQPQGPPSWTDECLSSQDEEHEADKKEDELEAMNAEEDSLRNGGEEEEEDE
DLEEEEEEEEEEEDQKPKRRGPKKKKMTKARLERFKLRRMKANARERNRMHGLNAALDNLRKVV
PCYSKTQKLSKIETLRLAKNYIWALSEILRSGKSPDLVSFVQTLCKGLSQPTTNLVAGCLQLNP
RTFLPEQNPDMPPHLPTASASFPVHPYSYQSPGLPSPPYGTMDSSHVFHVKPPPHAYSAALEPF
FESPLTDCTSPSFDGPLSPPLSINGNFSFKHEPSAEFEKNYAFTMHYPAATLAGPQSHGSIFSS
GAAAPRCEIPIDNIMSFDSHSHHERVMSAQLNAIFHD
SEQ ID NO: 5
Mouse LCN2 promoter
GCAGTGTGGAGACACACCCACTTTCCCCAAGGGCTCCTGCTCCCCCAAGTGATCCCCTTATCCT
CCGTGCTAAGATGACACCGAGGTTGCAGTCCTTACCTTTGAAAGCAGCCACAAGGGCGTGGGGG
TGCACACCTTTAATCCCAGCACTCGGGAGGCAGAGGCAGGCAGATTTCTGAGTTCGAGACCAGC
CTGGTCTACAAAGTGAATTCCAGGACAGCCAGGGCTATACAGAGAAACCCTGTCTTGAAAAAAA
AAGAGAAAGAAAAAAGAAAAAAAAAAATGAAAGCAGCCACATCTAAGGACTACGTGGCACAGGA
GAGGGTGAGTCCCTGAGAGTTCAGCTGCTGCCCTGTCTGTTCCTGTAAATGGCAGTGGGGTCAT
GGGAAAGTGAAGGGGCTCAAGGTATTGGACACTTCCAGGATAATCTTTTGGACGCCTCACCCTG
TGCCAGGACCAAGGCTGAGCTTGGCAGGCTCAGAACAGGGTGTCCTGTTCTTCCCTGTCTAAAA
CATTCACTCTCAGCTTGCTCACCCTTCCCCAGACAAGGAAGCTGCACAGGGTCTGGTGTTCAGA
TGGCTTTGGCTTACAGCAGGTGTGGGTGTGGGGTAGGAGGCAGGGGGTAGGGGTGGGGGAAGCC
TGTACTATACTCACTATCCTGTTTCTGACCCTCTAGGACTCCTACAGGGTTATGGGAGTGGACA
GGCAGTCCAGATCTGAGCTGCTGACCCACAAGCAGTGCCCTGTGCCTGCCAGAATCCAAAGCCC
TGGGAATGTCCCTCTGGTCCCCCTCTGTCCCCTGCAGCCCTTCCTGTTGCTCAACCTTGCACAG
TTCCGACCTGGGGGAGAGAGGGACAGAAATCTTGCCAAGTATTTCAACAGAATGTACTGGCAAT
TACTTCATGGCTTCCTGGACTTGGTAAAGGATGGACTACCCCGCCCAACAGGGGGGCTGGCAGC
CAGGTAGGCCCATAAAAAGCCCGCTGGGGAGTCCTCCTCACTCTCTGCTCTTCCTCCTCCAGCA
CACATCAGACCTAGTAGCTGTGGAAACCA
SEQ ID NO: 6
Human GFAP promoter
GTCTGCAAGCAGACCTGGCAGCATTGGGCTGGCCGCCCCCCAGGGCCTCCTCTTCATGCCCAGT
GAATGACTCACCTTGGCACAGACACAATGTTCGGGGTGGGCACAGTGCCTGCTTCCCGCCGCAC
CCCAGCCCCCCTCAAATGCCTTCCGAGAAGCCCATTGAGTAGGGGGCTTGCATTGCACCCCAGC
CTGACAGCCTGGCATCTTGGGATAAAAGCAGCACAGCCCCCTAGGGGCTGCCCTTGCTGTGTGG
CGCCACCGGCGGTGGAGAACAAGGCTCTATTCAGCCTGTGCCCAGGAAAGGGGATCAGGGGATG
CCCAGGCATGGACAGTGGGTGGCAGGGGGGGAGAGGAGGGCTGTCTGCTTCCCAGAAGTCCAAG
GACACAAATGGGTGAGGGGACTGGGCAGGGTTCTGACCCTGTGGGACCAGAGTGGAGGGCGTAG
ATGGACCTGAAGTCTCCAGGGACAACAGGGCCCAGGTCTCAGGCTCCTAGTTGGGCCCAGTGGC
TCCAGCGTTTCCAAACCCATCCATCCCCAGAGGTTCTTCCCATCTCTCCAGGCTGATGTGTGGG
AACTCGAGGAAATAAATCTCCAGTGGGAGACGGAGGGGTGGCCAGGGAAACGGGGCGCTGCAGG
AATAAAGACGAGCCAGCACAGCCAGCTCATGCGTAACGGCTTTGTGGAGCTGTCAAGGCCTGGT
CTCTGGGAGAGAGGCACAGGGAGGCCAGACAAGGAAGGGGTGACCTGGAGGGACAGATCCAGGG
GCTAAAGTCCTGATAAGGCAAGAGAGTGCCGGCCCCCTCTTGCCCTATCAGGACCTCCACTGCC
ACATAGAGGCCATGATTGACCCTTAGACAAAGGGCTGGTGTCCAATCCCAGCCCCCAGCCCCAG
AACTCCAGGGAATGAATGGGCAGAGAGCAGGAATGTGGGACATCTGTGTTCAAGGGAAGGACTC
CAGGAGTCTGCTGGGAATGAGGCCTAGTAGGAAATGAGGTGGCCCTTGAGGGTACAGAACAGGT
TCATTCTTCGCCAAATTCCCAGCACCTTGCAGGCACTTACAGCTGAGTGAGATAATGCCTGGGT
TATGAAATCAAAAAGTTGGAAAGCAGGTCAGAGGTCATCTGGTACAGCCCTTCCTTCCCTTTTT
TTTTTTTTTTTTTTGTGAGACAAGGTCTCTCTCTGTTGCCCAGGCTGGAGTGGCGCAAACACAG
CTCACTGCAGCCTCAACCTACTGGGCTCAAGCAATCCTCCAGCCTCAGCCTCCCAAAGTGCTGG
GATTACAAGCATGAGCCACCCCACTCAGCCCTTTCCTTCCTTTTTAATTGATGCATAATAATTG
TAAGTATTCATCATGGTCCAACCAACCCTTTCTTGACCCACCTTCCTAGAGAGAGGGTCCTCTT
GATTCAGCGGTCAGGGCCCCAGACCCATGGTCTGGCTCCAGGTACCACCTGCCTCATGCAGGAG
TTGGCGTGCCCAGGAAGCTCTGCCTCTGGGCACAGTGACCTCAGTGGGGTGAGGGGAGCTCTCC
CCATAGCTGGGCTGCGGCCCAACCCCACCCCCTCAGGCTATGCCAGGGGGTGTTGCCAGGGGCA
CCCGGGCATCGCCAGTCTAGCCCACTCCTTCATAAAGCCCTCGCATCCCAGGAGCGAGCAGAGC
CAGAGCAT
SEQ ID NO: 7
Mouse Aldh1L1 promoter
AACTGAGAGTGGAGGGGCACAGAAGAGCCCAAGAGGCTCCTTAGGTTGTGTGGAGGGTACAATA
TGTTTGGGCTGAGCAACCCAGAGCCAGACTTTGTCTGGCTGGTAAGAGACAGAGGTGCCTGCTA
TCACAATCCAAGGGTCTGCTTGAGGCAGAGCCAGTGCAAAGGATGTGGTTAGAGCCAGCCTGGT
GTACTGAAGAGGGGCGAAGAGCTTGAGTAAGGAGTCTCAGCGGTGGTTTGAGAGGCAGGGTGGT
TAATGGAGTAGCTGCAGGGGAGAATCCTTGGGAGGGAGCCTGCAGGACAGAGCTTTGGTCAGGA
AGTGATGGGCATGTCACTGGACCCTGTATTGTCTCTGACTTTTCTCAAGTAGGACAATGACTCT
GCCCAGGGAGGGGGTCTGTGACAAGGTGGAAGGGCCAGAGGAGAACTTCTGAGAAGAAAACCAG
AGGCCGTGAAGAGGTGGGAAGGGCATGGGATTCAGAACCTCAGGCCCACCAGGACACAACCCCA
GGTCCACAGCAGATGGGTGACCTTGCATGTCTCAGTCACCAGCATTGTGCTCCTTGCTTATCAC
GCTTGGGTGAAGGAAATGACCCAAATAGCATAAAGCCTGAAGGCCGGGACTAGGCCAGCTAGGG
CTTGCCCTTCCCTTCCCAGCTGCACTTTCCATAGGTCCCACCTTCAGCAGATTAGACCCGCCTC
CTGCTTCCTGCCTCCTTGCCTCCTCACTCATGGGTCTATGCCCACCTCCAGTCTCGGGACTGAG
GCTCACTGAAGTCCCATCGAGGTCTGGTCTGGTGAATCAGCGGCTGGCTCTGGGCCCTGGGCGA
CCAGTTAGGTTCCGGGCATGCTAGGCAATGAACTCTACCCGGAATTGGGGGTGCGGGGAGGCGG
GGAGGTCTCCAACCCAGCCTTTTGAGGACGTGCCTGTCGCTGCACGGTGCTTTTTATAGACGAT
GGTGGCCCATTTTGCAGAAGGGAAAGCCGGAGCCCTCTGGGGAGCAAGGTCCCCGCAAATGGAC
GGATGACCTGAGCTTGGTTCTGCCAGTCCACTTCCCAAATCCCTCACCCCATTCTAGGGACTAG
GGAAAGATCTCCTGATTGGTCATATCTGGGGGCCTGGCCGGAGGGCCTCCTATGATTGGAGAGA
TCTAGGCTGGGCGGGCCCTAGAGCCCGCCTCTTCTCTGCCTGGAGGAGGAGCACTGACCCTAAC
CCTCTCTGCACAAGACCCGAGCTTGTGCGCCCTTCTGGGAGCTTGCTGCCCCTGTGCTGACTGC
TGACAGCTGACTGACGCTCGCAGCTAGCAGGTACTTCTGGGTTGCTAGCCCAGAGCCCTGGGCC
GGTGACCCTGTTTTCCCTACTTCCCGTCTTTGACCTTGGGTAAGTTTCTTTTTCTTTTGTTTTT
GAGAGAGGCACCCAGATCCTCTCCACTACAGGCAGCCGCTGAACCTTGGATCCTCAGCTCCTGC
CCTGGGAACTACAGTTCCTGCCCTTTTTTTCCCACCTTGAGGGAGGTTTTCCCTGAGTAGCTTC
GACTATCCTGGAACAAGCTTTGTAGACCAGCCTGGGTCTCCGGAGAGTTGGGATTAAAGGCGTG
CACCACCACC
SEQ ID NO: 8
Human NG2 promoter
CTCTGGTTTCAAGACCAATACTCATAACCCCCACATGGACCAGGCACCATCACACCTGAGCACT
GCACTTAGGGTCAAAGACCTGGCCCCACATCTCAGCAGCTATGTAGACTAGCTCCAGTCCCTTA
ATCTCTCTCAGCCTCAGTTTCTTCATCTGCAAAACAGGTCTCAGTTTCGTTGCAAAGTATGAAG
TGCTGGGCTGTTACTGGTCAAAGGGAAGAGCTGGGAAGAGGGTGCAAGGTGGGGTTGGGCTGGA
GATGGGCTGGAGCAGATAGATGGAGGGACCTGAATGGAGGAAGTAAACCAAGGCCCGGTAACAT
TGGGACTGGACAGAGAACACGCAGATCCTCTAGGCACCGGAAGCTAAGTAACATTGCCCTTTCT
CCTCCTGTTTGGGACTAGGCTGATGTTGCTGCCTGGAAGGGAGCCAGCAGAAGGGCCCCAGCCT
GAAGCTGTTAGGTAGAAGCCAAATCCAGGGCCAGATTTCCAGGAGGCAGCCTCGGGAAGTTGAA
ACACCCGGATTCAGGGGTCAGGAGGCCTGGGCTTCTGGCACCAAACGGCCAGGGACCTACTTTC
CACCTGGAGTCTTGTAAGAGCCACTTTCAGCTTGAGCTGCACTTTCGTCCTCCATGAAATGGGG
GAGGGGATGCTCCTCACCCACCTTGCAAGGTTATTTTGAGGCAAATGTCATGGCGGGACTGAGA
ATTCTTCTGCCCTGCGAGGAAATCCAGACATCTCTCCCTTACAGACAGGGAGACTGAGGTGAGG
CCCTTCCAGGCAGAGAAGGTCACTGTTGCAGCCATGGGCAGTGCCCCACAGGACCTCGGGTGGT
GCCTCTGGAGTCTGGAGAAGTTCCTAGGGGACCTCCGAGGCAAAGCAGCCCAAAAGCCGCCTGT
GAGGGTGGCTGGTGTCTGTCCTTCCTCCTAAGGCTGGAGTGTGCCTGTGGAGGGGTCTCCTGAA
CTCCCGCAAAGGCAGAAAGGAGGGAAGTAGGGGCTGGGACAGTTCATGCCTCCTCCCTGAGGGG
GTCTCCCGGGCTCGGCTCTTGGGGCCAGAGTTCAGGGTGTCTGGGCCTCTCTATGACTTTGTTC
TAAGTCTTTAGGGTGGGGCTGGGGTCTGGCCCAGCTGCAAGGGCCCCCTCACCCCTGCCCCAGA
GAGGAACAGCCCCGCACGGGCCCTTTAAGAAGGTTGAGGGTGGGGGCAGGTGGGGGAGTCCAAG
CCTGAAACCCGAGCGGGCGCGCGGGTCTGCGCCTGCCCCGCCCCCGGAGTTAAGTGCGCGGACA
CCCGGAGCCGGCCCGCGCCCAGGAGCAGAGCCGCGCTCGCTCCACTCAGCTCCCAGCTCCCAGG
ACTCCGCTGGCTCCTCGCAAGTCCTGCCGCCCAGCCCGCCGGG
SEQ ID NO: 9
CAG::NeuroD1-IRES-GFP
GATCCGGCCATTAGCCATATTATTCATTGGTTATATAGCATAAATCAATATTGGCTATTGGCCA
TTGCATACGTTGTATCCATATCATAATATGTACATTTATATTGGCTCATGTCCAACATTACCGC
CATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAAC
GACCCCCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCA
TGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCATGTACGGTGGGAGGTCT
ATATAAGCAGAGCTCAATAAAAGAGCCCACAACCCCTCACTCGGGGCGCCAGTCCTCCGATTGA
CTGAGTCGCCCGGGTACCCGTATTCCCAATAAAGCCTCTTGCTGTTTGCATCCGAATCGTGGTC
TCGCTGTTCCTTGGGAGGGTCTCCTCTGAGTGATTGACTACCCACGACGGGGGTCTTTCATTTG
GGGGCTCGTCCGGGATTTGGAGACCCCTGCCCAGGGACCACCGACCCACCACCGGGAGGTAAGC
TGGCCAGCAACTTATCTGTGTCTGTCCGATTGTCTAGTGTCTATGTTTGATGTTATGCGCCTGC
GTCTGTACTAGTTAGCTAACTAGCTCTGTATCTGGCGGACCCGTGGTGGAACTGACGAGTTCTG
AACACCCGGCCGCAACCCTGGGAGACGTCCCAGGGACTTTGGGGGCCGTTTTTGTGGCCCGACC
TGAGGAAGGGAGTCGATGTGGAATCCGACCCCGTCAGGATATGTGGTTCTGGTAGGAGACGAGA
ACCTAAAACAGTTCCCGCCTCCGTCTGAATTTTTGCTTTCGGTTTGGAACCGAAGCCGCGCGTC
TTGTCTGCTGCAGCGCTGCAGCATCGTTCTGTGTTGTCTCTGTCTGACTGTGTTTCTGTATTTG
TCTGAAAATTAGGGCCAGACTGTTACCACTCCCTTAAGTTTGACCTTAGGTCACTGGAAAGATG
TCGAGCGGATCGCTCACAACCAGTCGGTAGATGTCAAGAAGAGACGTTGGGTTACCTTCTGCTC
TGCAGAATGGCCAACCTTTAACGTCGGATGGCCGCGAGACGGCACCTTTAACCGAGACCTCATC
ACCCAGGTTAAGATCAAGGTCTTTTCACCTGGCCCGCATGGACACCCAGACCAGGTCCCCTACA
TCGTGACCTGGGAAGCCTTGGCTTTTGACCCCCCTCCCTGGGTCAAGCCCTTTGTACACCCTAA
GCCTCCGCCTCCTCTTCCTCCATCCGCCCCGTCTCTCCCCCTTGAACCTCCTCGTTCGACCCCG
CCTCGATCCTCCCTTTATCCAGCCCTCACTCCTTCTCTAGGCGCCGGAATTCGATGTCGACATT
GATTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCCCA
TTGACGTCAATAATGACGTATGTTCCCATAGTAACGCCAATAGGGACTTTCCATTGACGTCAAT
GGGTGGACTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGC
GCGCGGCGGGCGGGAGTCGCTGCGTTGCCTTCGCCCCGTGCCCCGCTCCGCGCCGCCTCGCGCC
GCCCGCCCCGGCTCTGACTGACCGCGTTACTCCCACAGGTGAGCGGGCGGGACGGCCCTTCTCC
TCCGGGCTGTAATTAGCGCTTGGTTTAATGACGGCTCGTTTCTTTTCTGTGGCTGCGTGAAAGC
CTTAAAGGGCTCCGGGAGGGCCCTTTGTGCGGGGGGGAGCGGCTCGGGGGGTGCGTGCGTGTGT
GTGTGCGTGGGGAGCGCCGCGTGCGGCCCGCGCTGCCCGGCGGCTGTGAGCGCTGCGGGCGCGG
CGCGGGGCTTTGTGCGCTCCGCGTGTGCGCGAGGGGAGCGCGGCCGGGGGCGGTGCCCCGCGGT
GCGGGGGGGCTGCGAGGGGAACAAAGGCTGCGTGCGGGGTGTGTGCGTGGGGGGGTGAGCAGGG
GGTGTGGGCGCGGCGGTCGGGCTGTAACCCCCCCCTGCACCCCCCTCCCCGAGTTGCTGAGCAC
GGCCCGGCTTCGGGTGCGGGGCTCCGTGCGGGGCGTGGCGCGGGGCTCGCCGTGCCGGGCGGGG
GGTGGCGGCAGGTGGGGGTGCCGGGCGGGGCGGGGCCGCCTCGGGCCGGGGAGGGCTCGGGGGA
GGGGCGCGGCGGCCCCGGAGCGCCGGCGGCTGTCGAGGCGCGGCGAGCCGCAGCCATTGCCTTT
TATGGTAATCGTGCGAGAGGGCGCAGGGACTTCCTTTGTCCCAAATCTGGCGGAGCCGAAATCT
GGGAGGCGCCGCCGCACCCCCTCTAGCGGGCGCGGGCGAAGCGGTGCGGCGCCGGCAGGAAGGA
AATGGGCGGGGAGGGCCTTCGTGCGTCGCCGCGCCGCCGTCCCCTTCTCCATCTCCAGCCTCGG
GGCTGCCGCAGGGGGACGGCTGCCTTCGGGGGGGACGGGGCAGGGCGGGGTTCGGCTTCTGGCG
TGTGACCGGCGGCTCTAGAGCCTCTGCTAACCATGTTCATGCCTTCTTCTTTTTCCTACAGCTC
CTGGGCAACGTGCTGGTTGTTGTGCTGTCTCATCATTTTGGCAAAGAATTCGCTAGCGGATCCG
GCCGCCTCGGCCACCGGTCGCCACCATCGCCACCATGACCAAATCATACAGCGAGAGCGGGCTG
ATGGGCGAGCCTCAGCCCCAAGGTCCCCCAAGCTGGACAGATGAGTGTCTCAGTTCTCAGGACG
AGGAACACGAGGCAGACAAGAAAGAGGACGAGCTTGAAGCCATGAATGCAGAGGAGGACTCTCT
GAGAAACGGGGGAGAGGAGGAGGAGGAAGATGAGGATCTAGAGGAAGAGGAGGAAGAAGAAGAG
GAGGAGGAGGATCAAAAGCCCAAGAGACGGGGTCCCAAAAAGAAAAAGATGACCAAGGCGCGCC
TAGAACGTTTTAAATTAAGGCGCATGAAGGCCAACGCCCGCGAGCGGAACCGCATGCACGGGCT
GAACGCGGCGCTGGACAACCTGCGCAAGGTGGTACCTTGCTACTCCAAGACCCAGAAACTGTCT
AAAATAGAGACACTGCGCTTGGCCAAGAACTACATCTGGGCTCTGTCAGAGATCCTGCGCTCAG
GCAAAAGCCCTGATCTGGTCTCCTTCGTACAGACGCTCTGCAAAGGTTTGTCCCAGCCCACTAC
CAATTTGGTCGCCGGCTGCCTGCAGCTCAACCCTCGGACTTTCTTGCCTGAGCAGAACCCGGAC
ATGCCCCCGCATCTGCCAACCGCCAGCGCTTCCTTCCCGGTGCATCCCTACTCCTACCAGTCCC
CTGGACTGCCCAGCCCGCCCTACGGCACCATGGACAGCTCCCACGTCTTCCACGTCAAGCCGCC
GCCACACGCCTACAGCGCAGCTCTGGAGCCCTTCTTTGAAAGCCCCCTAACTGACTGCACCAGC
CCTTCCTTTGACGGACCCCTCAGCCCGCCGCTCAGCATCAATGGCAACTTCTCTTTCAAACACG
AACCATCCGCCGAGTTTGAAAAAAATTATGCCTTTACCATGCACTACCCTGCAGCGACGCTGGC
AGGGCCCCAAAGCCACGGATCAATCTTCTCTTCCGGTGCCGCTGCCCCTCGCTGCGAGATCCCC
ATAGACAACATTATGTCTTTCGATAGCCATTCGCATCATGAGCGAGTCATGAGTGCCCAGCTTA
ATGCCATCTTTCACGATTAGGTTTAAACGCGGCCGCGCCCCTCTCCCTCCCCCCCCCCTAACGT
TACTGGCCGAAGCCGCTTGGAATAAGGCCGGTGTGCGTTTGTCTATATGTTATTTTCCACCATA
TTGCCGTCTTTTGGCAATGTGAGGGCCCGGAAACCTGGCCCTGTCTTCTTGACGAGCATTCCTA
GGGGTCTTTCCCCTCTCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGGCAGCGGAACCCCCCA
CCTGGCGACAGGTGCCTCTGCGGCCAAAAGCCACGTGTATAAGATACACCTGCAAAGGCGGCAC
AACCCCAGTGCCACGTTGTGAGTTGGATAGTTGTGGAAAGAGTCAAATGGCTCTCCTCAAGCGT
ATTCAACAAGGGGCTGAAGGATGCCCAGAAGGTACCCCATTGTATGGGATCTGATCTGGGGCCT
CGGTGCACATGCTTTACATGTGTTTAGTCGAGGTTAAAAAAACGTCTAGGCCCCCCGAACCACG
GGGACGTGGTTTTCCTTTGAAAAACACGATGATAATATGGCCACAACCATGGTGAGCAAGGGCG
AGGAGCTGTTCACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGTAA
GTCGACAATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTAT
GGCTTTCATTTTCTCCTCCTTGTATAAATCCTGGTTGCTGTCTCTTTATGAGGAGTTGTGGCCC
GTTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTCC
GTGGTGTTGTCGGGGAAGCTGACGTCCTTTCCATGGCTGCTCGCCTGTGTTGCCACCTGGATTC
TGCGCGGGACGTCCTTCTGCTACGTCCCTTCGGCCCTCAATCCAGCGGACCTTCCTTCCCGCGG
CCTGCTGCCGGCTCTGCGGCCTCTTCCGCGTCTTCGCCTTCGCCCTCAGACGAGTCGGATCTCC
CTTTGGGCCGCCTCCCCGCCTGGAATTCGAGCTCGAGCTTGTTAACATCGATAAAATAAAAGAT
TTTATTTAGTCTCCAGAAAAAGGGGGGAATGAAAGACCCCACCTGTAGGTTTGGCAAGCTAGCT
TAAGTAACGCCATTTTGCAAGGCATGGAAAAATACATAACTGAGAATAGAGAAGTTCAGATCAA
GGTCAGGAACAGATGGAACAGCTGAATATGGGCCAAACAGGATATCTGTGGTAAGCAGTTCCTG
CCCCGGCTCAGGGCCAAGAACAGATGGAACAGCTGAATATGGGCCAAACAGGATATCTGTGGTA
AGCAGTTCCTGCCCCGGCTCAGGGCCAAGAACAGATGGTCCCCAGATGCGGTCCAGCCCTCAGC
AGTTTCTAGAGAACCATCAGATGTTTCCAGGGTGCCCCAAGGACCTGAAATGACCCTGTGCCTT
ATTTGAACTAACCAATCAGTTCGCTTCTCGCTTCTGTTCGCGCGCTTCTGCTCCCCGAGCTCAA
TAAAAGAGCCCACAACCCCTCACTCGGGGCGCCAGTCCTCCGATTGACTGAGTCGCCCGGGTAC
CCGTGTATCCAATAAACCCTCTTGCAGTTGCATCCGACTTGTGGTCTCGCTGTTCCTTGGGAGG
GTCTCCTCTGAGTGATTGACTACCCGTCAGCGGGGGTCTTTCATTTCCGACTTGTGGTCTCGCT
GCCTTGGGAGGGTCTCCTCTGAGTGATTGACTACCCGTCAGCGGGGGTCTTCACATGCAGCATG
TATCAAAATTAATTTGGTTTTTTTTCTTAAGTATTTACATTAAATGGCCATAGTTGCATTAATG
AATCGGCCAACGCGCGGGGAGAGGCGGTTTGCGTATTGGCGCTCTTCCGCTTCCTCGCTCACTG
ACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGT
GGCCTAACTACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGATTATC
AAAAAGGATCTTCACCTAGATCCTTTTAAATTAAAAATGAAGTTTGCGGCCGGCCGCAAATCAA
TCTAAAGTATATATGAGTAAACTTGGTCTGACAGTTACCAATGCTTAATCAGTGAGGCACCTAT
CTCAGCGATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTTTTCTG
TGACTGGTGAGTACTCAACCAAGTCATTCTGAGAATAGTGTATGCGGCGACCGAGTTGCTCTTG
CCCGGCGTCAACACGGGATAATACCGCGCCACATAGCAGAACTTTAAAAGTGCTCATCATTGGA
AAACGTTCTTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATA
CTCTTCCTTTTTCAAT
SEQ ID NO: 10
Human Dlx2 nucleic acid sequence encoding human Dlx2 protein
ATGACTGGAGTCTTTGACAGTCTAGTGGCTGATATGCACTCGACCCAGATCGCCGCCTCCAGCA
CGTACCACCAGCACCAGCAGCCCCCGAGCGGCGGCGGCGCCGGCCCGGGTGGCAACAGCAGCAG
CAGCAGCAGCCTCCACAAGCCCCAGGAGTCGCCCACCCTTCCGGTGTCCACCGCCACCGACAGC
AGCTACTACACCAACCAGCAGCACCCGGCGGGCGGCGGCGGCGGCGGGGGCTCGCCCTACGCGC
ACATGGGTTCCTACCAGTACCAAGCCAGCGGCCTCAACAACGTCCCTTACTCCGCCAAGAGCAG
CTATGACCTGGGCTACACCGCCGCCTACACCTCCTACGCTCCCTATGGAACCAGTTCGTCCCCA
GCCAACAACGAGCCTGAGAAGGAGGACCTTGAGCCTGAAATTCGGATAGTGAACGGGAAGCCAA
AGAAAGTCCGGAAACCCCGCACCATCTACTCCAGTTTCCAGCTGGCGGCTCTTCAGCGGCGTTT
CCAAAAGACTCAATACTTGGCCTTGCCGGAGCGAGCCGAGCTGGCGGCCTCTCTGGGCCTCACC
CAGACTCAGGTCAAAATCTGGTTCCAGAACCGCCGGTCCAAGTTCAAGAAGATGTGGAAAAGTG
GTGAGATCCCCTCGGAGCAGCACCCTGGGGCCAGCGCTTCTCCACCTTGTGCTTCGCCGCCAGT
CTCAGCGCCGGCCTCCTGGGACTTTGGTGTGCCGCAGCGGATGGCGGGCGGCGGTGGTCCGGGC
AGTGGCGGCAGCGGCGCCGGCAGCTCGGGCTCCAGCCCGAGCAGCGCGGCCTCGGCTTTTCTGG
GCAACTACCCCTGGTACCACCAGACCTCGGGATCCGCCTCACACCTGCAGGCCACGGCGCCGCT
GCTGCACCCCACTCAGACCCCGCAGCCGCATCACCACCACCACCATCACGGCGGCGGGGGCGCC
CCGGTGAGCGCGGGGACGATTTTCTAA
SEQ ID NO: 11
Human Dlx2 amino acid sequence - encoded by SEQ ID NO: 10
MTGVFDSLVADMHSTQIAASSTYHQHQQPPSGGGAGPGGNSSSSSSLHKPQESPTLPVSTATDS
SYYTNQQHPAGGGGGGGSPYAHMGSYQYQASGLNNVPYSAKSSYDLGYTAAYTSYAPYGTSSSP
ANNEPEKEDLEPEIRIVNGKPKKVRKPRTIYSSFQLAALQRRFQKTQYLALPERAELAASLGLT
QTQVKIWFQNRRSKFKKMWKSGEIPSEQHPGASASPPCASPPVSAPASWDFGVPQRMAGGGGPG
SGGSGAGSSGSSPSSAASAFLGNYPWYHQTSGSASHLQATAPLLHPTQTPQPHHHHHHHGGGGA
PVSAGTIF
SEQ ID NO: 12
Mouse Dlx2 nucleic acid sequence encoding mouse Dlx2 protein
ATGACTGGAGTCTTTGACAGTCTGGTGGCTGATATGCACTCGACCCAGATCACCGCCTCCAGCA
CGTACCACCAGCACCAGCAGCCCCCGAGCGGTGCGGGCGCCGGCCCTGGCGGCAACAGCAACAG
CAGCAGCAGCAACAGCAGCCTGCACAAGCCCCAGGAGTCGCCAACCCTCCCGGTGTCCACGGCT
ACGGACAGCAGCTACTACACCAACCAGCAGCACCCGGCGGGCGGCGGCGGCGGGGGGGCCTCGC
CCTACGCGCACATGGGCTCCTACCAGTACCACGCCAGCGGCCTCAACAATGTCTCCTACTCCGC
CAAAAGCAGCTACGACCTGGGCTACACCGCCGCGTACACCTCCTACGCGCCCTACGGCACCAGT
TCGTCTCCGGTCAACAACGAGCCGGACAAGGAAGACCTTGAGCCTGAAATCCGAATAGTGAACG
GGAAGCCAAAGAAAGTCCGGAAACCACGCACCATCTACTCCAGTTTCCAGCTGGCGGCCCTTCA
ACGACGCTTCCAGAAGACCCAGTATCTGGCCCTGCCAGAGCGAGCCGAGCTGGCGGCGTCCCTG
GGCCTCACCCAAACTCAGGTCAAAATCTGGTTCCAGAACCGCCGATCCAAGTTCAAGAAGATGT
GGAAAAGCGGCGAGATACCCACCGAGCAGCACCCTGGAGCCAGCGCTTCTCCTCCTTGTGCCTC
CCCGCCGGTCTCGGCGCCAGCATCCTGGGACTTCGGCGCGCCGCAGCGGATGGCTGGCGGCGGC
CCGGGCAGCGGAGGCGGCGGTGCGGGCAGCTCTGGCTCCAGCCCGAGCAGCGCCGCCTCGGCCT
TTCTGGGAAACTACCCGTGGTACCACCAGGCTTCGGGCTCCGCTTCACACCTGCAGGCCACAGC
GCCACTTCTGCATCCTTCGCAGACTCCGCAGGCGCACCATCACCACCATCACCACCACCACGCA
GGCGGGGGCGCCCCGGTGAGCGCGGGGACGATTTTCTAA
SEQ ID NO: 13
Mouse Dlx2 amino acid sequence - encoded by SEQ ID NO: 12
MTGVFDSLVADMHSTQITASSTYHQHQQPPSGAGAGPGGNSNSSSSNSSLHKPQESPTLPVSTA
TDSSYYTNQQHPAGGGGGGASPYAHMGSYQYHASGLNNVSYSAKSSYDLGYTAAYTSYAPYGTS
SSPVNNEPDKEDLEPEIRIVNGKPKKVRKPRTIYSSFQLAALQRRFQKTQYLALPERAELAASL
GLTQTQVKIWFQNRRSKFKKMWKSGEIPTEQHPGASASPPCASPPVSAPASWDFGAPQRMAGGG
PGSGGGGAGSSGSSPSSAASAFLGNYPWYHQASGSASHLQATAPLLHPSQTPQAHHHHHHHHHA
GGGAPVSAGTIF

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for (1) generating new glutamatergic neurons, (2) increasing survival of GABAergic neurons, (3) generating new non-reactive astrocytes, or (4) reducing the number of reactive astrocytes, in a mammal having had a hemorrhagic stroke and in need of (1), (2), (3), or (4), wherein said method comprises administering a composition comprising exogenous nucleic acid encoding a Neurogenic Differentiation 1 (NeuroD1) polypeptide or a biologically active fragment thereof and exogenous nucleic acid encoding a Distal-less homeobox 2 (Dlx2) polypeptide or a biologically active fragment thereof to said mammal.

2. The method of claim 1, wherein said mammal is a human.

3. The method of claim 1, wherein the hemorrhagic stroke is due to a condition selected from the group consisting of: ischemic stroke; physical injury; tumor; inflammation; infection; global ischemia as caused by cardiac arrest or severe hypotension (shock); hypoxic-ischemic encephalopathy as caused by hypoxia, hypoglycemia, or anemia; meningitis; and dehydration; or a combination of any two or more thereof.

4. The method of claim 1, wherein said administering step comprises delivering an expression vector comprising a nucleic acid encoding a NeuroD1 polypeptide or a biologically active fragment thereof and an expression vector comprising a nucleic acid encoding a Dlx2 polypeptide or a biologically active fragment thereof to the location of the hemorrhagic stroke in the brain.

5. The method of claim 1, wherein said administering step comprises delivering a recombinant viral expression vector comprising a nucleic acid encoding a NeuroD1 polypeptide or a biologically active fragment thereof and a recombinant viral expression vector comprising a nucleic acid encoding a Dlx2 polypeptide or a biologically active fragment thereof to the location of the hemorrhagic stroke in the brain.

6. The method of claim 1, wherein said administering step comprises delivering a recombinant adeno-associated virus expression vector comprising a nucleic acid encoding a NeuroD1 polypeptide or a biologically active fragment thereof and a recombinant adeno-associated virus expression vector comprising a nucleic acid encoding a Dlx2 polypeptide or a biologically active fragment thereof to the location of the hemorrhagic stroke in the brain.

7. The method of claim 1, wherein said administering step comprises a stereotactic intracranial injection to the location of the hemorrhagic stroke in the brain.

8. The method of claim 1, wherein said administering step further comprises administering the exogenous nucleic acid encoding a NeuroD1 polypeptide or a biologically active fragment thereof and exogenous nucleic acid encoding a Dlx2 polypeptide or a biologically active fragment thereof on one expression vector, one recombinant viral expression vector, or one recombinant adeno-associated virus expression vector.

9. The method of claim 1, wherein the composition comprises about 1 μL to about 500 μL of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof and a nucleic acid encoding a Dlx2 polypeptide or a biologically active fragment thereof.

10. The method of claim 9, wherein the composition is injected in the brain of said mammal at a controlled flow rate of about 0.1 μL/minute to about 5 μL/minute.

11. A method for (1) generating new GABAergic and glutamatergic neurons, (2) increasing survival of GABAergic and glutamatergic neurons, (3) generating new non-reactive astrocytes, or (4) reducing the number of reactive astrocytes, in a mammal having had a hemorrhagic stroke and in need of (1), (2), (3), or (4), wherein said method comprises administering a composition comprising exogenous nucleic acid encoding a Neurogenic Differentiation 1 (NeuroD1) polypeptide or a biologically active fragment thereof and exogenous nucleic acid encoding a Distal-less homeobox 2 (Dlx2) polypeptide or a biologically active fragment thereof to said mammal within 3 days of said hemorrhagic stroke.

12. The method of claim 11, wherein said mammal is a human.

13. The method of claim 11, wherein the hemorrhagic stroke is due to a condition selected from the group consisting of: bleeding in the brain; aneurysm; intracranial hematoma; subarachnoid hemorrhage; brain trauma; high blood pressure; weak blood vessels; malformation of blood vessels; ischemic stroke; physical injury; tumor; inflammation; infection; global ischemia as caused by cardiac arrest or severe hypotension (shock); hypoxic-ischemic encephalopathy as caused by hypoxia, hypoglycemia, or anemia; meningitis; and dehydration; or a combination of any two or more thereof.

14. The method of claim 11, wherein said administering step comprises delivering an expression vector comprising a nucleic acid encoding a NeuroD1 polypeptide or a biologically active fragment thereof and an expression vector comprising a nucleic acid encoding a Dlx2 polypeptide or a biologically active fragment thereof to the location of the hemorrhagic stroke in the brain.

15. The method of claim 11, wherein said administering step comprises delivering a recombinant viral expression vector comprising a nucleic acid encoding a NeuroD1 polypeptide or a biologically active fragment thereof and a recombinant viral expression vector comprising a nucleic acid encoding a Dlx2 polypeptide or a biologically active fragment thereof to the location of the hemorrhagic stroke in the brain.

16. The method of claim 11, wherein said administering step comprises delivering a recombinant adeno-associated virus expression vector comprising a nucleic acid encoding a NeuroD1 polypeptide or a biologically active fragment thereof and a recombinant adeno-associated virus expression vector comprising a nucleic acid encoding a Dlx2 polypeptide or a biologically active fragment thereof to the location of the hemorrhagic stroke in the brain.

17. The method of claim 11, wherein said administering step comprises a stereotactic intracranial injection to the location of the hemorrhagic stroke in the brain.

18. The method of claim 11, wherein said administering step further comprises administering the exogenous nucleic acid encoding a NeuroD1 polypeptide or a biologically active fragment thereof and exogenous nucleic acid encoding a Dlx2 polypeptide or a biologically active fragment thereof on one expression vector, one recombinant viral expression vector, or one recombinant adeno-associated virus expression vector.

19. The method of claim 11, wherein the composition comprises about 1 μL to about 500 L of a pharmaceutically acceptable carrier containing adeno-associated virus at a concentration of 10¹⁰-10¹⁴ adeno-associated virus particles/mL of carrier comprising a nucleic acid encoding a NeuroD1 polypeptide or a biologically active fragment thereof and a nucleic acid encoding a Dlx2 polypeptide or a biologically active fragment thereof.

20. The method of claim 19, wherein the composition is injected in the brain of said mammal at a controlled flow rate of about 0.1 μL/minute to about 5 μL/minute.
